(19) United States
(12) Patent Application Publication
(10) Pub. No.: US 2013/0209514 A1
(43) Pub. Date: Aug. 15, 2013

(54) APTAMER-TARGETED COSTIMULATORY LIGAND APTAMER

(71) Applicants: Eli Gilboa, Coral Gables, FL (US); Fernando Pastor, Coral Gables, FL (US)

(72) Inventors: Eli Gilboa, Coral Gables, FL (US); Fernando Pastor, Coral Gables, FL (US)

(21) Appl. No.: 13/376,873

(22) PCT Filed: Jun. 3, 2010
(86) PCT No.: PCT/US10/37164

§ 371 Date: Jun. 28, 2012

Related U.S. Application Data
(60) Provisional application No. 61/185,251, filed on Jun. 9, 2009.

(51) Int. Cl. A61K 39/00; A61K 39/385

(52) U.S. Cl. CPC A61K 39/0011; A61K 39/385
USPC 424/277.1; 530/358; 435/347

(57) ABSTRACT

Compositions for inducing or enhancing immunogenicity of a tumor comprise bi- and multi-specific aptamers binding to a tumor cell and an immune cell. These compositions have broad applicability in the treatment of many diseases, including cancer.

### CROSS-REFERENCE TO RELATED APPLICATIONS

The present application claims the priority of U.S. provisional patent application No. 61/185,251 filed Jun. 9, 2009, which is incorporated herein by reference in its entirety.

### SEQUENCE LISTING

The instant application contains a Sequence Listing which has been submitted via EFS-Web and is hereby incorporated by reference in its entirety. Said ASCII copy, created on Jul. 23, 2010, is named 7230116W.txt and is 3,809 bytes in size.

### FIELD OF THE INVENTION

Embodiments of the invention provide compositions and methods for highly selective targeting of heterologous nucleic acid sequences and delivery of a co-stimulatory and/or stimulatory signal to immune cells.

### BACKGROUND

Induction of potent anti-pathogen or anti-tumor immunity requires not only antigenic stimulation but also co-stimulation mediated by ligands which interact, with receptors on the surface of the immune cells, e.g. CD28, 4-1BB, OX40, etc. Tumor cells do not express costimulatory ligands and hence presentation of tumor antigens by the tumor cells does not potentiate the naturally occurring or vaccine-induced antitumor immune response. It was shown that provision of such costimulatory products to tumor cells enhances antitumor immunity and can lead to tumor regression. One way to provide costimulatory ligands to tumor cells, namely the tumor cells disseminated throughout the body of the patient, is to use soluble ligands or corresponding antibodies which bind to the costimulatory receptor. The problem with this approach is the well-known limitation of using protein-based therapeutics. A second approach is to use viral vectors such as pox or adenoviral vector to deliver the corresponding to gene to tumor cells in vivo. A major problem with this gene therapy approach is the complexity and cost of generating clinically approved reagents. Among other drawbacks, vectors such as for example, pox vectors only poorly penetrate solid tumors.

### SUMMARY

This Summary is provided to present a summary of the invention to briefly indicate the nature and substance of the invention. It is submitted with the understanding that it will not be used to interpret or limit the scope or meaning of the claims.

Embodiments of the invention comprise oligonucleotide-based aptamers which bind to a costimulatory target, for example, an aptamer which binds an immuno signaling molecule. The aptamer is targeted to disseminated tumor cells in vivo by linking to a second aptamer which recognizes a cell surface product expressed preferentially on tumor cells, for example, PSMA, which is expressed on prostate tumor cells.

In a preferred embodiment, the immune cells comprise T cells (T lymphocytes), B cells (B lymphocytes), antigen presenting cells, dendritic cells, monocytes, macrophages, myeloid suppressor cells, natural killer (NK) cells, cytotoxic T lymphocytes (CTLs), CTL lines, CTL clones, CTLs from tumor, inflammatory, or other infiltrates and subsets thereof. In some embodiments, the aptamer is specific for T lymphocytes and subsets thereof. Subsets of T lymphocytes are for example, T helper cells, CTLs, Treg.

In one embodiment, the aptamer is specific for immuno molecules comprising 4-1BB (CD137), OX40, CD3, CD28, CD27, CD70, CD270, TCR, CD28, CD137, CD137L, (Herpes Virus Entry Mediator (HVEM), TNFRSF14, ATAR, LIGHTR, TR2) HLA-ABC, HLA-DR, T Cell receptor αβ (TCRαβ), T Cell receptor γδ (TCRγδ), T cell receptor ζ (TCRζ), TGFβRII, TNF receptor, Cd11c, CD1-339, B7, mannose receptor, or DEC205, variants, mutants, species variants, ligands, alleles and fragments thereof.

Other aspects are described infra.

### BRIEF DESCRIPTION OF THE DRAWINGS

FIGS. 1A-1C show the functional characterization of a hi-specific PSMA-4-1BB aptamer conjugate. FIG. 1A: Sequence and computer generated secondary structure of the aptamer conjugate. A PSMA aptamer was annealed to the linker sequence of a 4-1BB aptamer dimer (see Methods for details). The RNAstructre 4.1 program was used for secondary structure analysis. FIG. 1B: Binding to PSMA-expressing CT26 tumor cells. Parental CT26 cells, CT26 cells expressing a wild type PSMA (PSMA-CT26) or CT26 cells expressing an internalization-deficient mutant (ΔPSMA-C126) were incubated with anti-PSMA antibody (green) or Cy3-conjugated PSMA-4-1BB aptamer conjugate (pink) and analyzed by confocal microscopy (40× magnification). Nuclei were stained with DAPI (blue), FIG. 1C: 4-1BB costimulation. CD8⁺ T cells were labeled with CFSE, activated with suboptimal concentrations of anti-CD3 antibody, and incubated with anti-4-1BB antibody/isotype control IgG, unconjugated agonistic 4-1BB/costimulation-deficient mut4-1BB aptamers, or with PSMA-4-1BB/PSMA-mut4-1BB aptamer diner conjugates. Two days later cells were analyzed by flow cytometry.

FIGS. 2A-2C show the inhibition of tumor growth in mice treated with PSMA-4-1BB aptamer conjugate. FIG. 2A: Subcutaneous tumor model. ΔPSMA-CT26 tumor cells were implanted subcutaneously in Balb/c mice (10 mice per group). At days 3, 4, 5 and 7 mice were injected via the tail vein with 50 pmoles of PSMA-4-1BB aptamer conjugate (▴), PSMA-mut4-1BB aptamer conjugate (▪) or with PBS () and monitored for tumor growth. FIG. 2B: Survival of mice shown in panel 2A, Mice were sacrificed when tumors reached 12 mm diameter. FIG. 2C: Lung metastasis model. C57BL/6 mice were implanted with ΔPSMA-B16/F10 cells via the tail vein (10 mice per group) and injected with PSMA-4-1BB or PSMA-mut4-1BB aptamer conjugates at days 5, 8, 11, 14. When about half of the mice in the control groups have shown signs of morbidity (circa days 25-28), mice were sacrificed and lungs were weighed.

FIG. 3 shows that PSMA targeted 4-1BB costimulation potentiates vaccine induced tumor immunity, C57B/6 mice were injected intravenously with B16.F10 tumor cells. At day 5 post tumor inoculation mice were treated with PSMA-4-1BB aptamer conjugates as described, in FIGS. 2A-2C except that the dose of aptamer conjugate was reduced to 25 pmoles per injection, and/or vaccinated with GM-CSF expressing irradiated B16/F10 tumor cells (GVAX). Lung metastasis was determined by measuring lung weight (left) and visual inspection (right).

FIGS. 4A, 4B: PSMA-4-1BB aptamer conjugate mediated inhibition of tumor growth is dependent on PSMA expression on the tumor cells. Balb/c were co-implanted subcutaneously with PSMA-expressing (left flank) and parental (right flank) CT26 tumor cells and injected with PSMA-4-1BB aptamer conjugate via the tail vein. FIG. 4A: 15 days post tumor inoculation ³²P-labeled aptamer conjugate was injected, and 6, 24 and 48 hours later tumors were excised and ³²P content determined (3 mice per group). FIG. 4B: 3 days post tumor inoculation mice were injected with PSMA-4-1BB or PSMA-mut4-1BB aptamer conjugates as described in FIG. 2A, or with 50 moles of unconjugated 4-1BB aptamer (5 mice per group) and tumor growth monitored. (◯) Parental C126, () ΔPSMA-CT26. Statistical analysis of average tumor size at day 19: ΔPSMA-CT26 versus CT26 tumor size in the PSMA-mut4-1BB treated mice, p=0.0051, and in the PSMA-41-BB treated mice, p=0.0013. ΔPSMA-CT26 tumor size in the PSMA-mut4-1BB versus PSMA-4-1BB treated mice, p=0.0007.

FIG. 5 shows the PSMA- and 4-1BB-dependent intratumoral infiltration of transgenic Pmel-1 CD8 T cells in mice treated with PSMA-4-1BB aptamer conjugate. B16/F10 (B16) or ΔPSMA-expressing B16/F10 (ΔPSMA-B16) tumor cells were implanted subcutaneously in C57BL/6 mice. Pmel-1 CD8 T cells which recognize an epitope of gp100, a tumor antigen expressed in B16/F10 tumor cells, were injected via the tail vein. At days 11, 12, 13, and 17 mice were injected with 50 pmoles of PSMA-4-1BB or PSMA-mut4-1BB aptamer conjugate or with PBS. At day 21 mice were sacrificed, tumor isolated, and tumor infiltrating Pmel-1 cells quantitated by flow cytometry. Where indicated, anti-4-1BB or isotype antibody was injected intratumorally immediately after aptamer injection.

FIGS. 6A-6B shows the therapeutic index of costimulatory 4-1BB ligands. FIG. 6A: Comparative analysis of the tumor inhibitory capacity of PSMA-4-1BB aptamer conjugate, free 4-1BB aptamer and 4-1BB antibody. Balb/c mice were implanted subcutaneously with ΔPSMA-C126 tumor cells and treated with PBS (◯), 50 (▾) or 500 (∇) pmoles of anti-4-1BB antibody, 50 (▪) or 500 (□) pmoles of unconjugated 4-1BB aptamer, or 50 pmoles PSMA-4-1BB aptamer conjugates (), starting at day 3 post tumor implantation as described in FIG. 2A (10 mice per group). Data were separated into two panels for clarity purposes, FIG. 6B: Evaluation of non-specific immune stimulatory effects in mice treated with therapeutic doses of 4-1BB ligands. Balb/c mice were injected with 4-1BB antibody (500 pmoles,) unconjugated 4-1BB aptamer (500 pmoles), PSMA-4-1BB aptamer conjugate (50 pmoles), or PBS at days 1, 2, 3, 5. Two weeks after the last injection mice were sacrificed, the spleen and the two inguinal lymph nodes were weighed and percentage of CD8 T cells in spleen and liver was determined by flow cytometry.

FIG. 7 is a scan of a photograph showing the coat discoloration in mice vaccinated with GVAX and treated with PSMA-4-1BB aptamer conjugates. See FIG. 3 for experimental details. Coat discoloration was seen in 3 out of 7 mice which also exhibited the most significant inhibition of metastasis as shown in FIG. 3. No coat discoloration was seen in mice from the other groups described in FIG. 3, or in mice treated with higher concentrations of PSMA-4-1BB aptamer alone which resulted in similar levels of metastasis inhibition (FIG. 2C).

FIG. 8 is a scan of a photograph showing the tumor size at day of sacrifice in mice co-implanted with PSMA-CT26 and parental CT26 tumor cells. Mice were sacrificed at day 19 (see experiment shown in FIGS. 4A, 4B) when the parental tumors reached maximum allowable size (>12 mm diameter). Only the PSMA-expressing, but not parental, CT26 tumors in mice treated with PSMA-4-1BB aptamer conjugate exhibited significant inhibition of growth at day 19; in three mice small tumors were palpable whereas in two mice tumors initially grew, became palpable, but fully regressed at the time of sacrifice.

### DETAILED DESCRIPTION

The present invention is described with reference to the attached figures, wherein like reference numerals are used throughout the figures to designate similar or equivalent elements. The figures are not drawn to scale and they are provided merely to illustrate the instant invention. Several aspects of the invention are described below with reference to example applications for illustration. It should be understood that numerous specific details, relationships, and methods are set forth to provide a fall understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### DEFINITIONS

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value, Where particular values are described in the application and claims, unless otherwise stated the term “about” meaning within an acceptable error range for the particular value should be assumed.

As used herein, a “target cell” or “recipient cell” refers to an individual cell or cell which is desired to be, or has been, bound by the aptamer-targeted costimulatory ligand aptamer. The term is also intended to include progeny of a single cell.

As used herein, the term “oligonucleotide,” includes linear or circular oligomers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. Oligonucleotides are capable of specifically binding to a target polynucleotide by way of a regular pattern of monomer-to-monomer interactions, such as Watson-Crick type of base pairing, Hoogsteen or reverse Hoogsteen types of base pairing, or the like.

The oligonucleotide may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide in the case of an oligonucleotide compound. These oligonucleotides typically comprise at least one region wherein the oligonucleotide is modified in order to exhibit one or more desired properties. The desired properties of the oligonucleotide include, but are not limited, for example, to increased resistance to nuclease degradation, increased cellular uptake, and/or increased binding affinity for the target nucleic acid. Different regions of the oligonucleotide may therefore have different properties. The chimeric oligonucleotides of the present invention can be formed as mixed structures of two or more oligonucleotides, modified oligonucleotides, oligonucleosides and/or oligonucleotide analogs as described above.

The oligonucleotide can be composed of regions that can be linked in “register,” that is, when the monomers are linked consecutively, as in native DNA, or linked via spacers. The spacers are intended to constitute a covalent “bridge” between the regions and have in preferred cases a length not exceeding about 100 carbon atoms. The spacers may carry different functionalities, for example, having positive or negative charge, carry special nucleic acid binding properties (intercalators, groove binders, toxins, fluorophors etc.), being lipophilic, inducing special secondary structures like, for example, alanine containing peptides that induce alpha-helices.

As used herein, the term “monomers” typically indicates monomers linked by phosphodiester bonds or analogs thereof to form oligonucleotides ranging in size from a few monomeric units, e.g., from about 3-4, to about several hundreds of monomeric units. Analogs of phosphodiester linkages include: phosphorothioate, phosphorodithioate, methylphosphomates, phosphoroselenoate, phosphoramidate, and the like, as more fully described below.

In the present context, the terms “nucleobase” covers naturally occurring nucleobases as well as non-naturally occurring nucleobases. It should be clear to the person skilled in the art that various nucleobases which previously have been considered “non-naturally occurring” have subsequently been found in nature. Thus, “nucleobase” includes not only the known purine and pyrimidine heterocycles, but also heterocyclic analogues and tautomers thereof. Illustrative examples of nucleobases are adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynylcytosine, 5-fluorouracil, 5-bromouracil, pseudoisocytosine, 2-hydroxy-5-methyl-4-triazolopyridin, isocytosine, isoguanin, inosine and the “non-naturally occurring” nucleobases described in Benner et al., U.S. Pat. No. 5,432,272. The term “nucleobase” is intended to cover every and all of these examples as well as analogues and tautomers thereof. Especially interesting nucleobases are adenine, guanine, thymine, cytosine, and uracil, which are considered as the naturally occurring nucleobases in relation to therapeutic and diagnostic application in humans.

As used herein, “nucleoside” includes the natural nucleosides, including 2′-deoxy and 2′-hydroxyl forms, e.g., as described in Kornberg and Baker, DNA Replication, 2nd Ed. (Freeman, San Francisco, 1992).

“Analogs” in reference to nucleosides includes synthetic nucleosides having modified base moieties and/or modified sugar moieties, e.g., described generally by Scheit, Nucleotide Analogs, John Wiley, New York, 1980; Freier & Altmann, Nucl. Acid. Res., 1997, 25(22), 4429-4443, Toulmé, J. J., Nature Biotechnology 19:17-18 (2001); Manoharan M., Biochemica et Biophysica Acta 1489:117-139 (1999); Freier S., M., Nucleic Acid Research, 25:4429-4443 (1997), Uhlman, E., Drug Discovery & Development, 3: 203-213 (2000), Herdewin P., Antisense & Nucleic Acid Drug Dev., 10:297-310 (2000),); 2′-O, 3′-C-linked [3.2.0]bicycloarabinonucleosides (see e.g. N. K Christiensen., et al, J. Am. Chem. Soc., 120: 5458-5463 (1998). Such analogs include synthetic nucleosides designed to enhance binding properties, e.g., duplex or triplex stability, specificity, or the like.

As used herein, the term “gene” means the gene and all currently known variants thereof and any further variants which may be elucidated. For example, when referring to a particular antigen, such as, for example, PSMA, the term refers to all variants, mutants, alleles, species etc.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type target gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs,) or single base mutations in which the polynucleotide sequence varies by one base. The presence of SNPs may be indicative of, for example, a certain population with a propensity for a disease state, that is susceptibility versus resistance.

As used herein, the term “mRNA” means the presently known mRNA transcript(s) of a targeted gene, and any further transcripts which may be elucidated.

The term, “complementary” means that two sequences are complementary when the sequence of one can bind to the sequence of the other in an anti-parallel sense wherein the 3′-end of each sequence binds to the 5′-end of the other sequence and each A, T(U), G, and C of one sequence is then aligned with a T(U), A, C, and G, respectively, of the other sequence. Normally, the complementary sequence of the oligonucleotide has at least 80% or 90%, preferably 95%, most preferably 100%, complementarity to a defined sequence. Preferably, alleles or variants thereof can be identified. A BLAST program also can be employed to assess such sequence identity.

The term “complementary sequence” as it refers to a polynucleotide sequence, relates to the base sequence in another nucleic acid molecule by the base-pairing rules. More particularly, the term or like term refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 95% of the nucleotides of the other strand, usually at least about 98%, and more preferably from about 99% to about 100%. Complementary polynucleotide sequences can be identified by a variety of approaches including use of well-known computer algorithms and software, for example the BLAST program.

The term “target nucleic acid” refers to a nucleic acid (often derived from a biological sample), to which the oligonucleotide is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA) whose expression level it is desired to detect. The difference in usage will be apparent from context.

By the term “modulate,” it is meant that any of the mentioned activities, are, e.g., increased, enhanced, increased, agonized (acts as an agonist), promoted, decreased, reduced, suppressed blocked, or antagonized (acts as an agonist). Modulation can increase activity more than 1-fold, 2-fold, 3-fold, 5-fold, 10-fold, 100-fold, etc., over baseline values. Modulation can also decrease its activity below baseline values. Modulation can also normalize an activity to a baseline value.

As used herein, a “pharmaceutically acceptable” component/carrier etc is one that is suitable for use with humans and/or animals without undue adverse side effects (such as toxicity, irritation, and allergic response) commensurate with a reasonable benefit/risk ratio.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer, either a sarcoma or lymphoma, or to shrink the cancer or prevent metastasis. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein, a “pharmaceutical salt” include, but are not limited to, mineral or organic acid salts of basic residues such as amines; alkali or organic salts of acidic residues such as carboxylic acids. Preferably the salts are made using an organic or inorganic acid. These preferred acid salts are chlorides, bromides, sulfates, nitrates, phosphates, sulfonates, formates, tartrates, maleates, malates, citrates, benzoates, salicylates, ascorbates, and the like. The most preferred salt is the hydrochloride salt.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy. Accordingly, “treating” or “treatment” of a state, disorder or condition includes: (1) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (2) inhibiting the state, disorder or condition, i.e., arresting, reducing or delaying the development of the disease or a relapse thereof (in case of maintenance treatment) or at least one clinical or subclinical symptom thereof; or (3) relieving the disease, i.e., causing regression of the state, disorder or condition or at least one of its clinical or subclinical symptoms. The benefit to an individual to be treated is either statistically significant or at least perceptible to the patient or to the physician.

“Target molecule” includes any macromolecule, including protein, carbohydrate, enzyme, polysaccharide, glycoprotein, receptor, antigen, antibody, growth factor; or it may be any small organic molecule including a hormone, substrate, metabolite, cofactor, inhibitor, drug, dye, nutrient, pesticide, peptide; or it may be an inorganic molecule including a metal, metal ion, metal oxide, and metal complex; it may also be an entire organism including a bacterium, virus, and single-cell eukaryote such as a protozoon.

In accordance with the present invention, there may be employed conventional molecular biology, microbiology, recombinant DNA, immunology, cell biology and other related techniques within the skill of the art. See, e.g., Sambrook et al., (2001) Molecular Cloning: A Laboratory Manual. 3^{rd} ed. Cold Spring Harbor Laboratory Press: Cold Spring Harbor, N.Y.; Sambrook et al., (1989) Molecular Cloning: A Laboratory Manual. 2^{nd} ed. Cold Spring Harbor Laboratory Press: Cold Spring Harbor, N.Y.; Ausubel et al., eds. (2005) Current Protocols in Molecular Biology. John Wiley and Sons, Inc.: Hoboken, N.J.; Bonifacino et al., eds. (2005) Current Protocols in Cell Biology. John Wiley and Sons, Inc.: Hoboken, N.J.; Coligan et al., eds. (2005) Current Protocols in Immunology, John Wiley and Sons, Inc.: Hoboken, N.J.; Coico et al., eds. (2005) Current Protocols in Microbiology, John Wiley and Sons, Inc.: Hoboken, N.J.; Coligan et al., eds. (2005) Current Protocols in Protein Science, John Wiley and Sons, Inc.: Hoboken, N.J.; Enna et al., eds. (2005) Current Protocols in Pharmacology John Wiley and Sons, Inc.: Hoboken, N.J.; Hames et al., eds. (1999) Protein Expression: A Practical Approach. Oxford University Press: Oxford; Freshney (2000) Culture of Animal Cells: A Manual of Basic Technique. 4^{th} ed. Wiley-Liss; among others. The Current Protocols listed above are updated several times every year.

### Compositions

Aptamers are oligonucleotide or peptide molecules that bind to a specific target molecule. Aptamers are usually created by selecting them from a large random sequence pool, but natural aptamers can also exist in riboswitches. More specifically, aptamers can be classified as: DNA or RNA aptamers and comprise strands of oligonucleotides. Peptide aptamers: comprising short variable peptide domains, attached at both ends to a protein scaffold.

Embodiments of the invention comprises the generation of oligonucleotide-based aptamers which bind to a costimulatory immune cell molecule and a cell surface product expressed preferentially on tumor cells, for example, a tumor antigen.

As used herein, the term “aptamer” refers to bi-specific or multi-specific molecules. For example, the aptamer can bind to two or more target cell antigens and two or more immune cell stimulatory and/or co-stimulatory antigens. The combinations of specificities can be determined by the user and as such, provides for unlimited combinations of specificities.

Co-stimulation of immune cells is mediated by ligands which interact with receptors on the surface of the immune cells, e.g. CD28, 4-1BB, OX40, etc. Tumor cells do not express costimulatory ligands and hence presentation of tumor antigens by the tumor cells does not potentiate the naturally occurring or a vaccine-induced antitumor immune response. As shown in the examples section which follows, the provision of such costimulatory products to tumor cells enhances antitumor immunity and can lead to tumor regression.

Studies in mice and cancer patients have shown that tumors are recognized by the immune system and can elicit an immune response which controls tumor progression. Yet, this naturally occurring tumor-induced immune response is weak and has a limited impact in delaying, but not reversing, tumor progression. A main reason why tumors are not “immunogenic” is that they don't express costimulatory ligands to promote the survival and expansion of the tumor-infiltrating T cells. In preferred embodiments, a clinically feasible and broadly useful composition and method to “coat” tumor cells with costimulatory ligands such as B7, 4-1BB or OX40 ligands, is provided herein.

As costimulatory ligands oligonucleotide aptamers bind and activate the cognate receptor. Aptamers with nuclease-resistant backbones exhibited remarkable affinity and specificity for their targets, comparable to and often exceeding that of antibodies. Unlike antibodies, aptamers or aptamer ODNs can be synthesized in a simple chemical process, offering a more straightforward and cost effective manufacturing and regulatory approval process for clinical use. To target the aptamer ligand to the tumors in vivo the costimulatory ligand aptamer was conjugated to a second aptamer which binds to a tumor-specific cell surface product.

The following example is provided merely for illustrative purposes and is not meant to limit or construe the application in any way. Briefly, a bispecific aptamer was generated and was composed of a 4-1BB binding aptamer conjugated to a PSMA binding aptamer. 4-1BB is a major costimulatory receptor expressed on CD8⁺ T cells and PSMA is a tumor specific product expressed on prostate tumor cells. The 4-1BB-PSMA bispecific aptamer bound to PSMA expressing tumor cells in vitro, in effect “coating” the tumor cells with the 4-1BB ligand. In vitro, the bispecific aptamer costimulated 4-1BB expressing CD8⁺ T cells promoting their proliferation and survival. The PSMA aptamer targeted delivery of the 4-1BB aptamer ligand to tumor cells in vivo inhibited tumor growth in the absence of other manipulations such as vaccination.

In preferred embodiments, the aptamer comprises at least one aptamer that specifically binds to an immune cell stimulatory molecule and a tumor antigen. Aptamers are oligonucleotide-based ligands that exhibit specificity and avidity comparable or superior to antibodies.

In a preferred embodiment, the compositions of the present invention are targeted to immune cell co-stimulatory molecules, for example, 4-1BB, CD27 (CD27 ligand is CD70), HVEM, LTβ receptors or ligands thereof.

In another preferred embodiments, the aptamer composition may comprise aptamer specific for one or more immune cell stimulatory molecules and one or more tumor antigens.

In yet another preferred embodiment, the aptamer compositions bind to two cells, an abnormal cell in which antigenicity is enhanced or up-regulated and an immune cell to effect a localized immune response. For example, if the abnormal cell is a tumor cell, the aptamer binds to a desired antigen and the immune cell thus bringing the two cells in contact. The advantage is that an immune response is localized. The aptamer serves to link the cells together.

The term “abnormal cell” refers to any cell which is not physiologically normal, for example, a tumor cell; a cell infected with an organism; transformed cell; a cell whereby the surface molecules are affected, such as, glycosylation or decrease in receptors etc; a cell which induces an autoimmune response; a cell which produces a mutant polynucleotide etc. Any cell which does not resemble a physiological or genetically normal cell would be considered an abnormal cell.

Immune System:

Immune systems are classified into two general systems, the “innate” or “primary” immune system and the “acquired/adaptive” or “secondary” immune system. It is thought that the innate immune system initially keeps the infection under control, allowing time for the adaptive immune system to develop an appropriate response. Studies have suggested that the various components of the innate immune system trigger and augment the components of the adaptive immune system, including antigen-specific B and T lymphocytes (Kos, Immunol. Res. 1998, 17:303; Romagnani, Immunol. Today. 1992, 13: 379; Banchereau and Steinman, Nature. 1988, 392:245).

A “primary immune response” refers to an innate immune response that is not affected by prior contact with the antigen. The main protective mechanisms of primary immunity are the skin (protects against attachment of potential environmental invaders), mucous (traps bacteria and other foreign material), gastric acid (destroys swallowed invaders), antimicrobial substances such as interferon (IFN) (inhibits viral replication) and complement proteins (promotes bacterial destruction), fever (intensifies action of interferons, inhibits microbial growth, and enhances tissue repair), natural killer (NK) cells (destroy microbes and certain tumor cells, and attack certain virus infected cells), and the inflammatory response (mobilizes leukocytes such as macrophages and dendritic cells to phagocytose invaders).

Some cells of the innate immune system, including macrophages and dendritic cells (DC), function as part of the adaptive immune system as well by taking up foreign antigens through pattern recognition receptors, combining peptide fragments of these antigens with major histocompatibility complex (MHC) class I and class II molecules, and stimulating naive CD8⁺ and CD4⁺ T cells respectively (Banchereau and Steinman, supra; Holmskov et al., Immunol. Today. 1994, 15:67; Ulevitch and Tobias Annu. Rev. Immunol. 1995, 13:437). Professional antigen-presenting cells (APCs) Communicate with these T cells, leading to the differentiation of naive CD4⁺ T cells into T-helper 1 (Th1) or T-helper 2 (Th2) lymphocytes that mediate cellular and humoral immunity, respectively (Trinchieri Annu. Rev. Immunol. 1995, 13:251; Howard and O'Garra, Immunol. Today. 1992, 13:198; Abbas et al., Nature. 1996, 383:787; Okamura et al., Adv. Immunol. 1998, 70:281; Mosmann and Sad, Immunol. Today. 1996, 17:138; O'Garra Immunity. 1998, 8:275).

A “secondary immune response” or “adaptive immune response” may be active or passive, and may be humoral (antibody based) or cellular that is established during the life of an animal, is specific for an inducing antigen, and is marked by an enhanced immune response on repeated encounters with said antigen. A key feature of the T lymphocytes of the adaptive immune system is their ability to detect minute concentrations of pathogen-derived peptides presented by MHC molecules on the cell surface. Upon activation, naïve CD4 T cells differentiate into one of at least two cell types, Th1 cells and Th2 cells, each type being characterized by the cytokines it produces. “Th1 cells” are primarily involved in activating macrophages with respect to cellular immunity and the inflammatory response, whereas “Th2 cells” or “helper T cells” are primarily involved in stimulating B cells to produce antibodies (humoral immunity). CD4 is the receptor for the human immunodeficiency virus (HIV). Effector molecules for Th1 cells include, but are not limited to, IFN-γ, GM-CSF, TNF-α, CD40 ligand, Fas ligand, IL-3, TNF-β, and IL-2. Effector molecules for Th2 cells include, but are not limited to, IL-4, IL-5, CD40 ligand, IL-3, GS-CSF, IL-10, TGF-β, and eotaxin. Activation of the Th1 type cytokine response can suppress the Th2 type cytokine response, and reciprocally, activation of the Th2 type cytokine response can suppress the Th1 type response.

In adaptive immunity, adaptive T and B cell immune responses work together with innate immune responses. The basis of the adaptive immune response is that of clonal recognition and response. An antigen selects the clones of cell which recognize it, and the first element of a specific immune response must be rapid proliferation of the specific lymphocytes. This is followed by further differentiation of the responding cells as the effector phase of the immune response develops. In T-cell mediated non-infective inflammatory diseases and conditions, immunosuppressive drugs inhibit T-cell proliferation and block their differentiation and effector functions.

The phrase “T cell response” means an immunological response involving T cells. The T cells that are “activated” divide to produce memory T cells or cytotoxic T cells. The cytotoxic T cells bind to and destroy cells recognized as containing the antigen. The memory T cells are activated by the antigen and thus provide a response to an antigen already encountered. This overall response to the antigen is the T cell response.

“Cells of the immune system” or “immune cells”, is meant to include any cells of the immune system that may be assayed, including, but not limited to, B lymphocytes, also called B cells, T lymphocytes, also called T cells, natural killer (NK) cells, natural killer T (NK) cells, lymphokine-activated killer (LAK) cells, monocytes, macrophages, neutrophils, granulocytes, mast cells, platelets, Langerhan's cells, stem cells, dendritic cells, peripheral blood mononuclear cells, tumor-infiltrating (TIL) cells, gene modified immune cells including hybridomas, drug modified immune cells, antigen presenting cells and derivatives, precursors or progenitors of the above cell types.

“Immune effector cells” refers to cells, and subsets thereof, e.g. Treg, Th1, Th2, capable of binding an antigen and which mediate an immune response selective for the antigen. These cells include, but are not limited to, T cells (T lymphocytes), B cells (B lymphocytes), antigen presenting cells, such as for example dendritic cells, monocytes, macrophages; myeloid suppressor cells, natural killer (NK) cells and cytotoxic T lymphocytes (CTLs), for example CTL lines, CTL clones, and CTLs from tumor, inflammatory, or other infiltrates.

A “T regulatory cell” or “Treg cell” or “Tr cell” refers to a cell that can inhibit a T cell response. Treg cells express the transcription factor Foxp3, which is not upregulated upon T cell activation and discriminates Tregs from activated effector cells. Tregs are identified by the cell surface markers CD25, CD45RB, CTLA4, and GITR. Treg development is induced by MSC activity. Several Treg subsets have been identified that have the ability to inhibit autoimmune and chronic inflammatory responses and to maintain immune tolerance in tumor-bearing hosts. These subsets include interleukin 10-(IL-10-) secreting T regulatory type 1 (Tr1) cells, transforming growth factor-β-(TGF-β-) secreting T helper type 3 (Th3) cells, and “natural” CD4⁺/CD25⁺ Tregs (Tm) (Fehervari and Sakaguchi. J. Clin. Invest. 2004, 114:1209-1217; Chen et al. Science. 1994, 265: 1237-1240; Groux et al. Nature. 1997, 389: 737-742).

The term “myeloid suppressor cell (MSC)” refers to a cell that is of hematopoietic lineage and expresses Gr-1 and CD11b; MSCs are also referred to as immature myeloid cells and were recently renamed to myeloid-derived suppressor cells (MDSCs). MSCs may also express CD 115 and/or F4/80 (see Li et al., Cancer Res. 2004, 64:1130-1139). MSCs may also express CD31, c-kit, vascular endothelial growth factor (VEGF)-receptor, or CD40 (Bronte et al., Blood. 2000, 96:3838-3846). MSCs may further differentiate into several cell types, including macrophages, neutrophils, dendritic cells, Langerhan's cells, monocytes or granulocytes. MSCs may be found naturally in normal adult bone marrow of human and animals or in sites of normal hematopoiesis, such as the spleen in newborn mice. Upon distress due to graft-versus-host disease (GVHD), cyclophosphamide injection, or γ-irradiation, for example, MSCs may be found in the adult spleen. MSCs can suppress the immunological response of T cells, induce T regulatory cells, and produce T cell tolerance. Morphologically, MSCs usually have large nuclei and a high nucleus-to-cytoplasm ratio. MSCs can secrete TFG-β and IL-10 and produce nitric oxide (NO) in the presence of IFN-γ or activated T cells. MSCs may form dendriform cells; however, MSCs are distinct from dendritic cells (DCs) in that DCs are smaller and express CD11c; MSCs do not express CD11c. T cell inactivation by MSCs in vitro can be mediated through several mechanisms: IFN-γ-dependent nitric oxide production (Kusmartsev et al. J Immunol. 2000, 165: 779-785); Th2-mediated-IL-4/IL-13-dependent arginase 1 synthesis (Bronte et al. J Immunol. 2003, 170: 270-278); loss of CD34 signaling in T cells (Rodriguez et al. J Immunol. 2003, 171: 1232-1239); and suppression of the T cell response through reactive oxygen species (Bronte et al. J Immunol. 2003, 170: 270-278; Bronte et al. Trends Immunol. 2003, 24: 302-306; Kusmartsev et al. J Immunol. 2004, 172: 989-999; Schmielau and Finn, Cancer Res. 2001, 61: 4756-4760).

Numerous costimulatory molecules have been identified playing a role in the initiation of immune responses by T and B lymphocytes. Signals provided through CD28-B7 interactions are essential for initial naïve T cell activation leading to increased IL-2 production and IL-2Rα (CD25) expression. NKG2D binds to the MHC-related proteins MIC and Rae-1 and induces IL-2 production and proliferation. In other cell types, such as B cells, activation requires CD40-CD40L interactions for proper antibody response: promoting survival, cytokine receptor expression, and inducing antibody class switch. In addition to the costimulatory pathways that are important in naïve lymphocyte activation, other costimulatory molecules play a role in effector/memory lymphocyte activation.

The costimulatory receptors ICOS, OX-40, 4-1BB, and CD27 bind to their ligands B7h, OX-40L, 4-1BBL, and CD70, respectively, to enhance the activation, survival, and cytokine secretion of effector/memory, but not naïve T and B cells. These costimulatory receptors and their ligands are not constitutively expressed but are induced on differentiated T cells, and their ligands are not restricted to APCs. T cell activation generally incorporates a self-limiting mechanism, such as inhibitory costimulators, to regulate T cell tolerance and attenuate the immune response. The expanding set of inhibitory costimulators currently includes CTLA-4 (CD152), PD-1, and BTLA. While expression of these molecules is induced following T cell activation, they are absent on naïve T cells. Lastly, B7-H3 is a new costimulatory ligand originally described to induce T cell proliferation and IFN-γ production through an as of yet unidentified receptor.

In preferred embodiments, the immune cell co-stimulatory induce an immune response. Examples of immune cell co-stimulatory molecules comprise: 4-1BB (CD137), OX40, CD2, CD3, CD4, CD8a, CD11a, CD11b, CD11c, CD19, CD20, CD25 (IL-2Rα), CD26, CD27, CD28, CD40, CD44, CD54, CD56, CD62L (L-Selectin), CD69 (VEA), CD70, CD80 (B7.1), CD83, CD86 (B7.2), CD95 (Fas), CD134 (OX-40), CD137, CD137L, (Herpes Virus Entry Mediator (HVEM), TNFRSF14, ATAR, LIGHTR, TR2), CD150 (SLAM), CD152 (CTLA-4), CD154, (CD40L), CD178 (FasL), CD209 (DC-SIGN), CD 270, CD277, AITR, AITRL, B7-H3, B7-H4, BTLA, HLA-ABC, HLA-DR, ICOS, ICOSL (B7RP-1), NKG2D, PD-1 (CD279), PD-L1 (B7-H1), PD-L2 (B7-DC), TCR-α, TCR-β, TCR-γ, TCR-δ, ZAP-70, lymphotoxin receptor (LTβ), NK 1.1, HLA-ABC, HLA-DR, T Cell receptor αβ (TCRαβ), T Cell receptor γδ (TCRγδ), T cell receptor ζ(TCRζ) TGFβRII, TNF receptor, Cd11c, CD1-339, B7, Foxp3, mannose receptor, or DEC205, variants, mutants, species variants, ligands, alleles and fragments thereof.

Examples of immune cells comprise T cells (T lymphocytes), B cells (B lymphocytes), antigen presenting cells, dendritic cells, monocytes, macrophages, myeloid suppressor cells, natural killer (NK) cells, NKT cells, NKT suppressor cells, T regulatory cells (Tregs), T suppressor cells, cytotoxic T lymphocytes (CTLs), CTL lines, CTL clones, CTLs from tumor, inflammatory, or other infiltrates and subsets thereof.

Natural killer T (NKT) cells are a heterogeneous group of T cells that share properties of both T cells and natural killer (NK) cells. Many of these cells recognize the non-polymorphic CD1d molecule, an antigen-presenting molecule that binds self- and foreign lipids and glycolipids. NKT cells are a subset of T cells that co-express an αβ T cell receptor (TCR), but also express a variety of molecular markers that are typically associated with NK cells, such as NK1.1. They differ from conventional αβ T cells in that their TCRs are far more limited in diversity and in that they recognize lipids and glycolipids presented by CD1d molecules, a member of the CD1 family of antigen presenting molecules, rather than peptide-MHC complexes. NKT cells include both NK1.1⁺ and NK1.1⁻, as well as CD4⁺, CD4⁻, CD8⁺ and CD8⁻ cells. Natural Killer T cells share other features with NK cells as well, such as CD16 and CD56 expression and granzyme production. NKT cells are classified into type I (invariant) and type II (non-invariant) cells in mice and humans. The best known subset of CD1d-dependent NKT cells expresses an invariant T cell receptor α (TCR-α) chain. These are referred to as type I or invariant NKT cells (iNKT) cells.

Originally called suppressor T cells (Ts cells), the most promising recent candidates have been termed regulatory T cells (Treg cells). Treg cells are a specialized subpopulation of T cells that act to suppress activation of the immune system and thereby maintain immune system homeostasis and tolerance to self-antigens. Regulatory T cells come in many forms, including those that express the CD8 transmembrane glycoprotein (CD8+ T cells), those that express CD4, CD25 and Foxp3 (CD4⁺CD25+ regulatory T cells or “Tregs”) and other T cell types that have suppressive function. These cells are involved in closing down immune responses after they have successfully tackled invading organisms, and also in keeping in check immune responses that may potentially attack one's own tissues (autoimmunity).

CD4⁺Foxp3⁺ regulatory T cells have been referred to as “naturally-occurring” regulatory T cells to distinguish them from “suppressor” T cell populations that are generated in vitro. Additional suppressive T cell populations, include Tr1, CD8⁺CD28⁻, and Qa-1 restricted T cells.

In preferred embodiments, an aptamer binds to one or more of the co-stimulatory molecules and one or more tumor cell antigens.

Potentiating Tumor Immunity Using Aptamer-Mediated Tumor Cell Immunogenicity:

Limited specificity of drugs and the need to reach all, or the vast majority, of the tumor cells disseminated throughout the body are the two major challenges in developing effective treatments for cancer. Mechanistic studies of tumorigenesis at the molecular and cellular levels have stimulated new paradigms of increasingly sophisticated large-scale drug screening programs. A complementary, and a more general, approach to increase the immunogenicity of cancer cells.

Immune responses in cancer patients are often far from ideal. Since cancer cells are altered-self cells, one would expect cancerous cells to elicit a cell-mediated response. There are three processes that must occur for tumor elimination. The immune system must recognize the tumor cell, activate lymphocytes, and the cancer cells must be susceptible to killing. In order for this to take place, lymphocytes should be able to infiltrate to the tumor site. CD4⁺ T-helper1 (Th1) lymphocytes should then recognize tumor-specific antigens in association with MHC II molecules on the surface of professional antigen presenting cells and receive signals from costimulatory molecules such as B7. As a result, Th1 lymphocytes should be activated and release appropriate cytokines including interleukin-2, interferon-gamma and tumor necrosis factor-alpha. These cytokines, in addition to stimulation by tumor-specific antigens presented on cancer cell surface MHC I molecules, should activate cytotoxic “killer” T lymphocytes (CTLs) to lysis cancerous cells. B lymphocytes should also be activated to secrete neutralizing antibodies that aid in cancer cell phagocytosis by antigen presenting cells, although their role in tumor immunity is less important. CTL-mediated lysis of a cancerous cell, the ultimate action of an effective immune response against cancer, is shown below. If any of the processes necessary for the induction of a cell-mediated response fail, tumor elimination may not be effective.

Some immune evasion strategies that prevent antigenicity of cancer cells are described below.

Tissue Localization (Sequestration):

There are several sites in the body, such as the central nervous system, which are inaccessible to the immune system. Tumors in such areas of the body are invisible to immune surveillance and thus cannot be targeted by immune reactions. Residence of a tumor in immune privileged sites allows them to be essentially non-antigenic because the immune system is not even aware of their presence.

The immunogenicity of a tumor antigen also seems to be affected by the location of the tumor antigen. Fibroblasts, which lack costimulatory molecules and cytokines, can activate T cells only after a few of them have drained into the vicinity of the lymphoid organs. In other sites in the body, small numbers of fibroblasts are non-immunogenic and can therefore go unnoticed. This indicates that, depending on the carcinogen, a tumor may or may not go unnoticed in a particular area of the body. Since many tumor cells lack costimulatory properties, they can only be detected if they are in the appropriate environment within the body. Cancer cells that manage to avoid the lymphoid organs may be able to sneak through and develop into large tumors. After having reached this stage, it is extremely difficult for the immune system to effectively combat the tumor. This phenomenon is similar to the idea of sequestration in that the tumor may be positioned in a place where the immune system will not mount an immune response against it due to the fact that in that particular location, the numbers of tumor cells are not great enough to be immunogenic.

Thus, the aptamer molecule which is designed to specifically bind a tumor antigen and an immune cell co-stimulatory antigen, essentially coats the tumor cell with these aptamer molecules which can then bind and activate the cognate receptor, thus stimulating an immune response and increasing the immunogenicity of a tumor cell.

Antigenic Modulation:

Cancer cells can readily alter themselves to evade immunologic recognition and attack. Tumor cells alter their characteristics to evade attack by the immune system. They are capable of generating variants lacking features that mark them for destruction by T cells, killer cells and antibodies. This process is called antigenic modulation or immunoselection.

The aptamers described herein, can be generated to be specific for antigens on tumor cells which would not ordinarily be recognized by the immune cells. Furthermore, the aptamers can be generated to more than one tumor antigens.

Lack of Costimulation:

Melanoma tumor cells are immunogenic; theoretically, they should cause an immune response but they do not stimulate an effective anti-tumor immune response in vivo. Melanoma tumors may be capable of delivering antigen-specific signals to T cells, but do not deliver the costimulatory signals necessary for full activation of T cells because of the lack of B7 expression on their surface. T cell activation requires two distinct signaling events. The first signal originates from the binding of the TCR to its antigen-MHC ligand, and provides the specificity of the interaction. The second signal is either provided by soluble factors such as IL-2 or the interaction of cell-surface molecules on the T cell with their ligands on APCs. This second signal is thought to provide the necessary costimulation to the TCR-mediated signaling event. Binding of the TCR with peptide-MHC complexes in the absence of costimulation can result in T cell inactivation or anergy, which is associated with a block in the IL-2 gene transcription.

For example, expression of B7 on the surface of a cell is the costimulatory signal necessary to allow for the cytolytic CD8⁺ T cell attack on the tumor. The costimulation results from an interaction of the CD28 molecule on the T cell surface with its ligand, B7, on the surface of an antigen-presenting cell (APC). B7 display renders tumor cells capable of effective antigen presentation, leading to their eventual eradication.

In preferred embodiments, enhancing or inducing the immunogenicity of a tumor cell in vivo comprises administering to a patient a composition comprising a bi- or multi-specific aptamer which binds to tumor antigens and immune cell co-stimulatory and/or stimulatory molecules and eliciting an immune cell response specific for that tumor or any other target, such as for example, virus infected cell. Thus, the aptamer composition modulates the functions of the cells, for example, proliferation of a lymphocyte wherein that lymphocyte had been previously suppressed or attenuated.

The cell can be any type of one or more immune cells. In some preferred embodiments, the immune cell is a lymphocyte. These reagents or compositions involved or associated with modulating immunity, such as costimulation (i.e., CTLA-4, 4-1BB, PD-1, etc.) serve as important adjunct to, or replace altogether, new and powerful, often complex, vaccination protocols currently under development.

In another preferred embodiment, the bi- or multi-specific aptamer compositions target cells involved in rendering the immune system tolerant to a particular antigen or antigens. “Tolerance” refers to the anergy (non-responsiveness) of immune cells, e.g. T cells, when presented with an antigen. T cell tolerance prevents a T cell response even in the presence of an antigen that existing memory T cells recognize.

In another preferred embodiment, the aptamers can be used in to treating any disease wherein immunogenicity of a target is desired, for example, viral diseases.

In preferred embodiments, the oligonucleotides can be tailored to individual therapy, for example, these oligonucleotides can be sequence specific for allelic variants in individuals, the up-regulation in immunogenicity of a target can be manipulated in varying degrees, such as for example, 10%, 20%, 40%, 100% expression relative to the control. That is, in some patients it may be effective to increase immunogenicity by 10% versus 80% in another patient.

Immunogenicity of a target can be monitored by various techniques known in the art such as, immuno assays, blotting, and the like.

Aptamer Composition:

By “aptamer” or “nucleic acid aptamer” as used herein is meant a nucleic acid molecule that binds specifically to a target molecule wherein the nucleic acid molecule has sequence that comprises a sequence recognized by the target molecule in its natural setting. Alternately, an aptamer can be a nucleic acid molecule that binds to a target molecule wherein the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art (see, e.g., Gold et al., Annu. Rev. Biochem. 64:763, 1995; Brody and Gold, J. Biotechnol. 74:5, 2000; Sun, Curr. Opin. Mol. Ther. 2:100, 2000; Kusser, J. Biotechnol. 74:27, 2000; Hermann and Patel, Science 287:820, 2000; and Jayasena, Clinical Chem. 45:1628, 1999).

The aptamer may be linked to one or more other aptamers with similar or varying specificities by a linker. A non-nucleotide linker may be comprised of an abasic nucleotide, polyether, polyamine, polyamide, peptide, carbohydrate, lipid, polyhydrocarbon, or other polymeric compounds (e.g., polyethylene glycols such as those having between 2 and 100 ethylene glycol units). Specific examples include those described by Seela and Kaiser, Nucleic Acids Res. 18:6353, 1990, and Nucleic Acids Res. 15:3113, 1987; Cload and Schepartz, J. Am. Chem. Soc. 113:6324, 1991; Richardson and Schepartz, J. Am. Chem. Soc. 113:5109, 1991; Ma et al., Nucleic Acids Res. 21:2585, 1993, and Biochemistry 32:1751, 1993; Durand et al., Nucleic Acids Res. 18:6353, 1990; McCurdy et al., Nucleosides & Nucleotides 10:287, 1991; Jaschke et al., Tetrahedron Lett. 34:301, 1993; Ono et al., Biochemistry 30:9914, 1991; Arnold et al., PCT Publication No. WO 89/02439; Usman et al., PCT Publication No. WO 95/06731; Dudycz et al., PCT Publication No. WO 95/11910 and Ferentz and Verdine, J. Am. Chem. Soc. 113:4000, 1991.

The invention may be used against protein coding gene products as well as non-protein coding gene products. Examples of non-protein coding gene products include gene products that encode ribosomal RNAs, transfer RNAs, small nuclear RNAs, small cytoplasmic RNAs, telomerase RNA, RNA molecules involved in DNA replication, chromosomal rearrangement and the like.

In another preferred embodiment, the nucleobases in the aptamers may be modified to provided higher specificity and affinity for a target. For example nucleobases may be substituted with LNA monomers, which can be in contiguous stretches or in different positions. The modified molecules, preferably have a higher association constant (Kₐ) for the target sequences than the complementary sequence. Binding of the modified or non-modified molecules to target sequences can be determined in vitro under a variety of stringency conditions using hybridization assays.

Certain preferred aptamer oligonucleotides of this invention are chimeric oligonucleotides. “Chimeric oligonucleotides” or “chimeras,” in the context of this invention, are oligonucleotides which contain two or more chemically distinct regions, each made up of at least one nucleotide. These oligonucleotides typically contain at least one region of modified nucleotides that confers one or more beneficial properties, such as, for example, increased nuclease resistance, increased binding affinity for the target molecule. Consequently, comparable results can often be obtained with shorter oligonucleotides when chimeric oligonucleotides are used, compared to phosphorothioate deoxyoligonucleotides hybridizing to the same target region.

In one preferred embodiment, a chimeric oligonucleotide comprises at least one region modified to increase target binding affinity. Affinity of an oligonucleotide for its target (in this case, a nucleic acid encoding ras) is routinely determined by measuring the Tₘ of an oligonucleotide/target pair, which is the temperature at which the oligonucleotide and target dissociate; dissociation is detected spectrophotometrically. The higher the Tₘ, the greater the affinity of the oligonucleotide for the target.

In another preferred embodiment, the region of the oligonucleotide which is modified comprises at least one nucleotide modified at the 2′ position of the sugar, preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrymidines, abasic residues or an inverted base at the 3′ end of the RNA. Such modifications are routinely incorporated into oligonucleotides and these oligonucleotides have been shown to have a higher Tₘ (i.e., higher target binding affinity) than; T-deoxyoligonucleotides against a given target. The effect of such increased affinity is to greatly enhance RNAi oligonucleotide inhibition of gene expression. RNAse H is a cellular endonuclease that cleaves the RNA strand of RNA:DNA duplexes; activation of this enzyme therefore results in cleavage of the RNA target, and thus can greatly enhance the efficiency of RNAi inhibition. Cleavage of the RNA target can be routinely demonstrated by gel electrophoresis. In another preferred embodiment, the chimeric oligonucleotide is also modified to enhance nuclease resistance. Cells contain a variety of exo- and endo-nucleases which can degrade nucleic acids. A number of nucleotide and nucleoside modifications have been shown to make the oligonucleotide into which they are incorporated more resistant to nuclease digestion than the native oligodeoxynucleotide.

Nuclease resistance is routinely measured by incubating oligonucleotides with cellular extracts or isolated nuclease solutions and measuring the extent of intact oligonucleotide remaining over time, usually by gel electrophoresis. Oligonucleotides which have been modified to enhance their nuclease resistance survive intact for a longer time than unmodified oligonucleotides. A variety of oligonucleotide modifications have been demonstrated to enhance or confer nuclease resistance. Oligonucleotides which contain at least one phosphorothioate modification are presently more preferred. In some cases, oligonucleotide modifications which enhance target binding affinity are also, independently, able to enhance nuclease resistance. Some desirable modifications can be found in De Mesmaeker et al. Acc. Chem. Res. 1995, 28:366-374.

Specific examples of some preferred oligonucleotides envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH, —N(CH₃)—O—CH₂ [known as a methylene(methylimino) or MMI backbone], CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH₁). The amide backbones disclosed by De Mesmaeker et al. Acc. Chem. Res. 1995, 28:366-374) are also preferred. Also preferred are oligonucleotides having morpholino backbone structures (Summerton and Weller, U.S. Pat. No. 5,034,506). In other preferred embodiments, such as the peptide nucleic acid (PNA) backbone, the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). Oligonucleotides may also comprise one or more substituted sugar moieties. Preferred oligonucleotides comprise one of the following at the 2′ position: OH, SH, SCH₃, F, OCN, OCH₃OCH₃, OCH₃O(CH₂)ₙCH₃, O(CH₂)ₙNH₂ or O(CH₂)ₙCH₃ where n is from 1 to about 10; C₁ to C₁₀ lower alkyl, alkoxyalkoxy, substituted lower alkyl, alkaryl or aralkyl; Cl; Br; CN; CF₃; OCF₃; O—, S—, or N-alkyl; O—, S—, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂CH₂OCH₃, also known as 2′-O-(2-methoxyethyl)] (Martin et al., Helv. Chim. Acta, 1995, 78, 486). Other preferred modifications include 2′-methoxy (2′-O—CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Oligonucleotides may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N₆ (6-aminohexyl)adenine and 2,6-diaminopurine. Kornberg, A., DNA Replication, W. H. Freeman & Co., Sari Francisco, 1980, pp 75-T7; Gebeyehu, G., et al. Nucl. Acids Res. 1987, 15:4513). A “universal” base known in the art, e.g., inosine, may be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278) and are presently preferred base substitutions.

Another modification of the oligonucleotides of the invention involves chemically linking to the oligonucleotide one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-5-tritylthiol (Manoharan et al. Ann. N.Y. Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651). Oligonucleotides comprising lipophilic moieties, and methods for preparing such oligonucleotides are known in the art, for example, U.S. Pat. Nos. 5,138,045, 5,218,105 and 5,459,255.

It is not necessary for all positions in a given oligonucleotide to be uniformly modified, and in fact more than one of the aforementioned modifications may be incorporated in a single oligonucleotide or even at within a single nucleoside within an oligonucleotide. The present invention also includes oligonucleotides which are chimeric oligonucleotides as hereinbefore defined.

In another embodiment, the nucleic acid molecules are conjugated with other moieties including but not limited to abasic nucleotides, polyether, polyamine, polyamides, peptides, carbohydrates, lipid, or polyhydrocarbon compounds. Those skilled in the art will recognize that these molecules can be linked to one or more of any nucleotides comprising the nucleic acid molecule at several positions on the sugar, base or phosphate group.

The oligonucleotides used in accordance with this invention may be conveniently and routinely made through the well-known technique of solid phase synthesis. Equipment for such synthesis is sold by several vendors including Applied Biosystems. Any other means for such synthesis may also be employed; the actual synthesis of the oligonucleotides is well within the talents of one of ordinary skill in the art. It is also well known to use similar techniques to prepare other oligonucleotides such as the phosphorothioates and alkylated derivatives. It is also well known to use similar techniques and commercially available modified amidites and controlled-pore glass (CPG) products such as biotin, fluorescein, acridine or psoralen-modified amidites and/or CPG (available from Glen Research, Sterling Va.) to synthesize fluorescently labeled, biotinylated or other modified oligonucleotides such as cholesterol-modified oligonucleotides.

In accordance with the invention, use of modifications such as the use of LNA monomers to enhance the potency, specificity and duration of action and broaden the routes of administration of oligonucleotides comprised of current chemistries such as MOE, ANA, FANA, PS etc (Recent advances in the medical chemistry of antisense oligonucleotide by Uhlman, Current Opinions in Drug Discovery & Development 2000 Vol 3 No 2). This can be achieved by substituting some of the monomers in the current oligonucleotides by LNA monomers. The LNA modified oligonucleotide may have a size similar to the parent compound or may be larger or preferably smaller. It is preferred that such LNA-modified oligonucleotides contain less than about 70%, more preferably less than about 60%, most preferably less than about 50% LNA monomers and that their sizes are between about 10 and 25 nucleotides, more preferably between about 12 and 20 nucleotides.

### Generation of Aptamers

Aptamers are high affinity single-stranded nucleic acid ligands which can be isolated from combinatorial libraries through an iterative process of in vitro selection known as SELEX™ (Systemic Evolution of Ligands by EXponential enrichment). Aptamers exhibit specificity and avidity comparable to or exceeding that of antibodies, and can be generated against most targets. Unlike antibodies, aptamers, can be synthesized in a chemical process and hence offer significant advantages in terms of reduced production cost and much simpler regulatory approval process. Also, aptamers are not expected to exhibit significant immunogenicity in vivo.

In preferred embodiments, at least one aptamer is linked to at least one other aptamer which is specific for a desired cell antigen and a stimulatory and/or co-stimulatory immune cell target molecule. In other embodiments, a plurality of aptamers can be directed to different target molecules and stimulatory and/or co-stimulatory molecules. The various permutations and combinations for combining aptamers is limited only by the imagination of the user.

Methods of the present disclosure do not require a priori knowledge of the nucleotide sequence of every possible gene variant (including mRNA splice variants) targeted.

Aptamers specific for a given biomolecule can be identified using techniques known in the art. See, e.g., Toole et al. (1992) PCT Publication No. WO 92/14843; Tuerk and Gold (1991) PCT Publication No. WO 91/19813; Weintraub and Hutchinson (1992) PCT Publication No. 92/05285; and Ellington and Szostak, Nature 346:818 (1990). Briefly, these techniques typically involve the complexation of the molecular target with a random mixture of oligonucleotides. The aptamer-molecular target complex is separated from the uncomplexed oligonucleotides. The aptamer is recovered from the separated complex and amplified. This cycle is repeated to identify those aptamer sequences with the highest affinity for the molecular target.

The SELEX™ process is a method for the in vitro evolution of nucleic acid molecules with highly specific binding to target molecules and is described in, e.g., U.S. Pat. No. 5,270,163 (see also WO 91/19813) entitled “Nucleic Acid Ligands”. Each SELEX-identified nucleic acid ligand is a specific ligand of a given target compound or molecule. The SELEX™ process is based on the unique insight that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets.

SELEX™ relies as a starting point upon a large library of single stranded oligonucleotides comprising randomized sequences derived from chemical synthesis on a standard DNA synthesizer. The oligonucleotides can be modified or unmodified DNA, RNA or DNA/RNA hybrids. In some examples, the pool comprises 100% random or partially random oligonucleotides. In other examples, the pool comprises random or partially random oligonucleotides containing at least one fixed sequence and/or conserved sequence incorporated within randomized sequence. In other examples, the pool comprises random or partially random oligonucleotides containing at least one fixed sequence and/or conserved sequence at its 5′ and/or 3′ end which may comprise a sequence shared by all the molecules of the oligonucleotide pool. Fixed sequences are sequences common to oligonucleotides in the pool which are incorporated for a pre-selected purpose such as, CpG motifs, hybridization sites for PCR primers, promoter sequences for RNA polymerases (e.g., T3, T4, T7, and SP6), restriction sites, or homopolymeric sequences, such as poly A or poly T tracts, catalytic cores, sites for selective binding to affinity columns, and other sequences to facilitate cloning and/or sequencing of an oligonucleotide of interest. Conserved sequences are sequences, other than the previously described fixed sequences, shared by a number of aptamers that bind to the same target.

The oligonucleotides of the pool preferably include a randomized sequence portion as well as fixed sequences necessary for efficient amplification. Typically the oligonucleotides of the starting pool contain fixed 5′ and 3′ terminal sequences which flank an internal region of 30-50 random nucleotides. The randomized nucleotides can be produced in a number of ways including chemical synthesis and size selection from randomly cleaved cellular nucleic acids. Sequence variation in test nucleic acids can also be introduced or increased by mutagenesis before or during the selection/amplification iterations.

The random sequence portion of the oligonucleotide can be of any length and can comprise ribonucleotides and/or deoxyribonucleotides and can include modified or non-natural nucleotides or nucleotide analogs. See, e.g., U.S. Pat. No. 5,958,691; U.S. Pat. No. 5,660,985; U.S. Pat. No. 5,958,691; U.S. Pat. No. 5,698,687; U.S. Pat. No. 5,817,635; U.S. Pat. No. 5,672,695, and PCT Publication WO 92/07065. Random oligonucleotides can be synthesized from phosphodiester-linked nucleotides using solid phase oligonucleotide synthesis techniques well known in the art. See, e.g., Froehler et al., Nucl. Acid Res. 14:5399-5467 (1986) and Froehler et al., Tet. Lett. 27:5575-5578 (1986). Random oligonucleotides can also be synthesized using solution phase methods such as triester synthesis methods. See, e.g., Sood et al., Nucl. Acid Res. 4:2557 (1977) and Hirose et al., Tet. Lett., 28:2449 (1978). Typical syntheses carried out on automated DNA synthesis equipment yield 10¹⁴-10¹⁶ individual molecules, a number sufficient for most SELEX™ experiments. Sufficiently large regions of random sequence in the sequence design increases the likelihood that each synthesized molecule is likely to represent a unique sequence.

The starting library of oligonucleotides may be generated by automated chemical synthesis on a DNA synthesizer. To synthesize randomized sequences, mixtures of all four nucleotides are added at each nucleotide addition step during the synthesis process, allowing for random incorporation of nucleotides. As stated above, in one embodiment, random oligonucleotides comprise entirely random sequences; however, in other embodiments, random oligonucleotides can comprise stretches of nonrandom or partially random sequences. Partially random sequences can be created by adding the four nucleotides in different molar ratios at each addition step.

The starting library of oligonucleotides may be either RNA or DNA. In those instances where an RNA library is to be used as the starting library it is typically generated by transcribing a DNA library in vitro using T7 RNA polymerase or modified T7 RNA polymerases and purified. The RNA or DNA library is then mixed with the target under conditions favorable for binding and subjected to step-wise iterations of binding, partitioning and amplification, using the same general selection scheme, to achieve virtually any desired criterion of binding affinity and selectivity. More specifically, starting with a mixture containing the starting pool of nucleic acids, the SELEX™ method includes steps of: (a) contacting the mixture with the target under conditions favorable for binding; (b) partitioning unbound nucleic acids from those nucleic acids which have bound specifically to target molecules; (c) dissociating the nucleic acid-target complexes; (d) amplifying the nucleic acids dissociated from the nucleic acid-target complexes to yield a ligand-enriched mixture of nucleic acids; and (e) reiterating the steps of binding, partitioning, dissociating and amplifying through as many cycles as desired to yield highly specific, high affinity nucleic acid ligands to the target molecule. In those instances where RNA aptamers are being selected, the SELEX™ method further comprises the steps of: (i) reverse transcribing the nucleic acids dissociated from the nucleic acid-target complexes before amplification in step (d); and (ii) transcribing the amplified nucleic acids from step (d) before restarting the process.

Within a nucleic acid mixture containing a large number of possible sequences and structures, there is a wide range of binding affinities for a given target. A nucleic acid mixture comprising, for example, a 20 nucleotide randomized segment can have 4²⁰ candidate possibilities. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands or aptamers.

Cycles of selection and amplification are repeated until a desired goal is achieved. In the most general case, selection/amplification is continued until no significant improvement in binding strength is achieved on repetition of the cycle. The method is typically used to sample approximately 10¹⁴ different nucleic acid species but may be used to sample as many as about 10¹⁸ different nucleic acid species. Generally, nucleic acid aptamer molecules are selected in a 5 to 20 cycle procedure. In one embodiment, heterogeneity is introduced only in the initial selection stages and does not occur throughout the replicating process. In one embodiment of SELEX™, the selection process is so efficient at isolating those nucleic acid ligands that bind most strongly to the selected target, that only one cycle of selection and amplification is required. Such an efficient selection may occur, for example, in a chromatographic-type process wherein the ability of nucleic acids to associate with targets bound on a column operates in such a manner that the column is sufficiently able to allow separation and isolation of the highest affinity nucleic acid ligands.

In many cases, it is not necessarily desirable to perform the iterative steps of SELEX™ until a single nucleic acid ligand is identified. The target-specific nucleic acid ligand solution may include a family of nucleic acid structures or motifs that have a number of conserved sequences and a number of sequences which can be substituted or added without significantly affecting the affinity of the nucleic acid ligands to the target. By terminating the SELEX™ process prior to completion, it is possible to determine the sequence of a number of members of the nucleic acid ligand solution family.

A variety of nucleic acid primary, secondary and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. Almost all known cases of such motifs suggest that they can be formed in a nucleic acid sequence of no more than 30 nucleotides. For this reason, it is often preferred that SELEX™ procedures with contiguous randomized segments be initiated with nucleic acid sequences containing a randomized segment of between about 20 to about 50 nucleotides and in some embodiments, about 30 to about 40 nucleotides. In one example, the 5′-fixed:random:3′-fixed sequence comprises a random sequence of about 30 to about 50 nucleotides.

The core SELEX™ method can be modified to achieve a number of specific objectives. For example, U.S. Pat. No. 5,707,796 describes the use of SELEX™ in conjunction with gel electrophoresis to select nucleic acid molecules with specific structural characteristics, such as bent DNA. U.S. Pat. No. 5,763,177 describes SELEX™ based methods for selecting nucleic acid ligands containing photo reactive groups capable of binding and/or photo-cross linking to and/or photo-inactivating a target molecule. U.S. Pat. No. 5,567,588 and U.S. Pat. No. 5,861,254 describe SELEX™ based methods which achieve highly efficient partitioning between oligonucleotides having high and low affinity for a target molecule. U.S. Pat. No. 5,496,938 describes methods for obtaining improved nucleic acid ligands after the SELEX™ process has been performed. U.S. Pat. No. 5,705,337 describes methods for covalently linking a ligand to its target. SELEX™ can also be used to obtain nucleic acid ligands that bind to more than one site on the target molecule, and to obtain nucleic acid ligands that include non-nucleic acid species that bind to specific sites on the target.

Counter-SELEX™ is a method for improving the specificity of nucleic acid ligands to a target molecule by eliminating nucleic acid ligand sequences with cross-reactivity to one or more non-target molecules. Counter-SELEX™ is comprised of the steps of: (a) preparing a candidate mixture of nucleic acids; (b) contacting the candidate mixture with the target, wherein nucleic acids having an increased affinity to the target relative to the candidate mixture may be partitioned from the remainder of the candidate mixture; (c) partitioning the increased affinity nucleic acids from the remainder of the candidate mixture; (d) dissociating the increased affinity nucleic acids from the target; (e) contacting the increased affinity nucleic acids with one or more non-target molecules such that nucleic acid ligands with specific affinity for the non-target molecule(s) are removed; and (f) amplifying the nucleic acids with specific affinity only to the target molecule to yield a mixture of nucleic acids enriched for nucleic acid sequences with a relatively higher affinity and specificity for binding to the target molecule. As described above for SELEX™, cycles of selection and amplification are repeated as necessary until a desired goal is achieved.

One potential problem encountered in the use of nucleic acids as therapeutics and vaccines is that oligonucleotides in their phosphodiester form may be quickly degraded in body fluids by intracellular and extracellular enzymes such as endonucleases and exonuclease before the desired effect is manifest. The SELEX™ method thus encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. For example, oligonucleotides containing nucleotide derivatives chemically modified at the 2′ position of ribose, 5 position of pyrimidines, and 8 position of purines, 2′-modified pyrimidines, nucleotides modified with 2′-amino (2′—NH₂), 2′-fluoro (2′-F), and/or 2′-O-methyl (2′-OMe) substituents.

In preferred embodiments, one or more modifications of the nucleic acid ligands contemplated in this invention include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Modifications to generate oligonucleotide populations which are resistant to nucleases can also include one or more substitute internucleotide linkages, altered sugars, altered bases, or combinations thereof. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, and unusual base-pairing combinations such as the isobases isocytidine and isoguanosine. Modifications can also include 3′ and 5′ modifications such as capping.

In one embodiment, oligonucleotides are provided in which the P(O)0 group is replaced by P(O)S (“thioate”), P(S)S (“dithioate”), P(O)NR₂ (“amidate”), P(O)R, P(O)OR′, CO or CH₂ (“formacetal”) or 3′-amine (—NH—CH₂—CH₂—), wherein each R or R′ is independently H or substituted or unsubstituted alkyl. Linkage groups can be attached to adjacent nucleotides through an —O—, —N—, or —S— linkage. Not all linkages in the oligonucleotide are required to be identical. As used herein, the term phosphorothioate encompasses one or more non-bridging oxygen atoms in a phosphodiester bond replaced by one or more sulfur atom.

In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al., Nucl. Acid Res. 19:2629-2635 (1991); and Hobbs, et al., Biochemistry 12:5138-5145 (1973). Other modifications are known to one of ordinary skill in the art. Such modifications may be pre-SELEX™ process modifications or post-SELEX™ process modifications (modification of previously identified unmodified ligands) or may be made by incorporation into the SELEX™ process.

Pre-SELEX™ process modifications or those made by incorporation into the SELEX™ process yield nucleic acid ligands with both specificity for their SELEX™ target and improved stability, e.g., in vivo stability. Post-SELEX™ process modifications made to nucleic acid ligands may result in improved stability, e.g., in vivo stability without adversely affecting the binding capacity of the nucleic acid ligand.

The SELEX™ method encompasses combining selected oligonucleotides with other selected oligonucleotides and non-oligonucleotide functional units as described in U.S. Pat. No. 5,637,459 and U.S. Pat. No. 5,683,867. The SELEX™ method further encompasses combining selected nucleic acid ligands with lipophilic or non-immunogenic high molecular weight compounds in a diagnostic or therapeutic complex, as described, e.g., in U.S. Pat. No. 6,011,020, U.S. Pat. No. 6,051,698, and PCT Publication No. WO 98/18480. These patents and applications teach the combination of a broad array of shapes and other properties, with the efficient amplification and replication properties of oligonucleotides, and with the desirable properties of other molecules.

The identification of nucleic acid ligands to small, flexible peptides via the SELEX™ method can also be used in embodiments of the invention. Small peptides have flexible structures and usually exist in solution in an equilibrium of multiple conformers.

The aptamers with specificity and binding affinity to the target(s) of the present invention are typically selected by the SELEX™ process as described herein. As part of the SELEX™ process, the sequences selected to bind to the target can then optionally be minimized to determine the minimal sequence having the desired binding affinity. The selected sequences and/or the minimized sequences are optionally optimized by performing random or directed mutagenesis of the sequence to increase binding affinity or alternatively to determine which positions in the sequence are essential for binding activity. Additionally, selections can be performed with sequences incorporating modified nucleotides to stabilize the aptamer molecules against degradation in vivo.

Further aptamers can be obtained using various methods. In a preferred embodiment, a variation of the SELEX™ process is used to discover aptamers that are able to enter cells or the sub-cellular compartments within cells. These delivery aptamers will allow or increase the propensity of an oligonucleotide to enter or be taken up by a cell. The method comprises the ability to selectively amplify aptamers that have been exposed to the interior of a cell and became modified in some fashion as a result of that exposure. Such modifications include functioning as a template for template-dependent polymerization. This variation of SELEX™ permits the discovery of aptamers that are: (i) completely specific with regard to the kind of cell or sub-cellular compartment, such as the nucleus or cytoplasm, that they permit entry to, (ii) completely generic, or (iii) partially specific.

One potential strategy is to substitute cell-association for cell entry, and after incubation of the library with the cells and subsequent washing of the cells, amplify the library members that remain associated with the cells. However, this may not distinguish between aptamers that permit genuine cell entry and other trivial solutions to the cell-association problem such as binding to the exterior of the cell membrane, entering, but not leaving, the cell membrane and being taken up by, but not leaving, the endosome.

An alternative strategy is to select for some kind of transformation of the oligonucleotide library member that could happen only in the cytoplasm or other sub-cellular compartment, optionally because the library member is conjugated to a transformable entity, and then selectively amplifying the transformed library members. Such markers include, but are not limited to: reverse transcription, RNaseH, kinase, 5′-phosphorylation, 5′-dephosphorylation, translation-dependent, post-transcriptional modification to give restrictable cDNA, transcription-based, ubiquitination, ultracentrifugation, or utilizing the endogenous protein kinase Clp1. For example, library members can have a designed hairpin structure at their 3′-terminus that will reverse-transcribe without a primer. Reverse transcriptase activity is introduced into the cytoplasm using a protein expression vector or virus. The selective amplification of reverse-transcribed sequences is achieved by using a nucleotide composition that will not amplify directly by, for example, PCR such as completely or partially 2′-OH or 2′OMe RNA and omitting an RT step from the procedure.

In yet another aspect, aptamers that selectively bind to variants of target gene expression products can be identified, e.g. new tumor antigens, or other types of desired antigen or stimulatory molecule targets. A “variant” is an alternative form of a gene. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

Sequence similarity searches can be performed manually or by using several available computer programs known to those skilled in the art. Preferably, Blast and Smith-Waterman algorithms, which are available and known to those skilled in the art, and the like can be used. Blast is NCBI's sequence similarity search tool designed to support analysis of nucleotide and protein sequence databases. Blast can be accessed through the world wide web of the Internet, at, for example, ncbi.nlm.nih.gov/BLAST/. The GCG Package provides a local version of Blast that can be used either with public domain databases or with any locally available searchable database. GCG Package v9.0 is a commercially available software package that contains over 100 interrelated software programs that enables analysis of sequences by editing, mapping, comparing and aligning them. Other programs included in the GCG Package include, for example, programs which facilitate RNA secondary structure predictions, nucleic acid fragment assembly, and evolutionary analysis. In addition, the most prominent genetic databases (GenBank, EMBL, PIR, and SWISS-PROT) are distributed along with the GCG Package and are fully accessible with the database searching and manipulation programs. GCG can be accessed through the Internet at, for example, http://www.gcg.com/. Fetch is a tool available in GCG that can get annotated GenBank records based on accession numbers and is similar to Entrez. Another sequence similarity search can be performed with GeneWorld and GeneThesaurus from Pangea. GeneWorld 2.5 is an automated, flexible, high-throughput application for analysis of polynucleotide and protein sequences. GeneWorld allows for automatic analysis and annotations of sequences. Like GCG, GeneWorld incorporates several tools for homology searching, gene finding, multiple sequence alignment, secondary structure prediction, and motif identification. GeneThesaurus 1.0™ is a sequence and annotation data subscription service providing information from multiple sources, providing a relational data model for public and local data.

Another alternative sequence similarity search can be performed, for example, by BlastParse. BlastParse is a PERL script running on a UNIX platform that automates the strategy described above. BlastParse takes a list of target accession numbers of interest and parses all the GenBank fields into “tab-delimited” text that can then be saved in a “relational database” format for easier search and analysis, which provides flexibility. The end result is a series of completely parsed GenBank records that can be easily sorted, filtered, and queried against, as well as an annotations-relational database.

In accordance with the invention, paralogs can be identified for designing the appropriate aptamers. Paralogs are genes within a species that occur due to gene duplication, but have evolved new functions, and are also referred to as isotypes.

The polynucleotides of this invention can be isolated using the technique described in the experimental section or replicated using PCR. The PCR technology is the subject matter of U.S. Pat. Nos. 4,683,195, 4,800,159, 4,754,065, and 4,683,202 and described in PCR: The Polymerase Chain Reaction (Mullis et al. eds, Birkhauser Press, Boston (1994)) and references cited therein. Alternatively, one of skill in the art can use the identified sequences and a commercial DNA synthesizer to replicate the DNA. Accordingly, this invention also provides a process for obtaining the polynucleotides of this invention by providing the linear sequence of the polynucleotide, nucleotides, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can insert the polynucleotide into a suitable replication vector and insert the vector into a suitable host cell (prokaryotic or eukaryotic) for replication and amplification. The DNA so amplified can be isolated from the cell by methods well known to those of skill in the art. A process for obtaining polynucleotides by this method is further provided herein as well as the polynucleotides so obtained.

Another suitable method for identifying targets for the aptamer compositions includes contacting a test sample with a cell expressing a receptor or gene thereof, an allele or fragment thereof; and detecting interaction of the test sample with the gene, an allele or fragment thereof, or expression product of the gene, an allele or fragment thereof. The desired gene, an allele or fragment thereof, or expression product of the gene, an allele or fragment thereof suitably can be detectably labeled e.g. with a fluorescent or radioactive component.

In another preferred embodiment, a cell from a patient is isolated and contacted with a drug molecule that modulates an immune response. The genes, expression products thereof, are monitored to identify which genes or expression products are regulated by the drug. Aptamers can then be synthesized to regulate the identified genes, expression products that are regulated by the drug and thus, provide therapeutic oligonucleotides. These can be tailored to individual patients, which is advantageous as different patients do not effectively respond to the same drugs equally. Thus, the oligonucleotides would provide a cheaper and individualized treatment than conventional drug treatments.

In one aspect, hybridization with oligonucleotide probes that are capable of detecting polynucleotide sequences, including genomic sequences, encoding desired genes or closely related molecules may be used to identify target nucleic acid sequences. The specificity of the probe, whether it is made from a highly specific region, e.g., the 5′ regulatory region, or from a less specific region, e.g., a conserved motif, and the stringency of the hybridization or amplification (maximal, high, intermediate, or low), will determine whether the probe identifies only naturally occurring sequences encoding genes, allelic variants, or related sequences.

Probes may also be used for the detection of related sequences, and should preferably have at least 50% sequence identity or homology to any of the identified genes encoding sequences, more preferably at least about 60, 70, 75, 80, 85, 90 or 95 percent sequence identity to any of the identified gene encoding sequences (sequence identity determinations discussed above, including use of BLAST program). The hybridization probes of the subject invention may be DNA or RNA and may be derived from the sequences of the invention or from genomic sequences including promoters, enhancers, and introns of the gene.

Identity of genes, or variants thereof, can be verified using techniques well known in the art. Examples include but are not limited to, nucleic acid sequencing of amplified genes, hybridization techniques such as single nucleic acid polymorphism analysis (SNP), microarrays wherein the molecule of interest is immobilized on a biochip. Overlapping cDNA clones can be sequenced by the dideoxy chain reaction using fluorescent dye terminators and an ABI sequencer (Applied Biosystems, Foster City, Calif.). Any type of assay wherein one component is immobilized may be carried out using the substrate platforms of the invention. Bioassays utilizing an immobilized component are well known in the art. Examples of assays utilizing an immobilized component include for example, immunoassays, analysis of protein-protein interactions, analysis of protein-nucleic acid interactions, analysis of nucleic acid-nucleic acid interactions, receptor binding assays, enzyme assays, phosphorylation assays, diagnostic assays for determination of disease state, genetic profiling for drug compatibility analysis, SNP detection, etc.

Identification of a nucleic acid sequence capable of binding to a biomolecule of interest can be achieved by immobilizing a library of nucleic acids onto the substrate surface so that each unique nucleic acid was located at a defined position to form an array. The array would then be exposed to the biomolecule under conditions which favored binding of the biomolecule to the nucleic acids. Non-specifically binding biomolecules could be washed away using mild to stringent buffer conditions depending on the level of specificity of binding desired. The nucleic acid array would then be analyzed to determine which nucleic acid sequences bound to the biomolecule. Preferably the biomolecules would carry a fluorescent tag for use in detection of the location of the bound nucleic acids.

An assay using an immobilized array of nucleic acid sequences may be used for determining the sequence of an unknown nucleic acid; single nucleotide polymorphism (SNP) analysis; analysis of gene expression patterns from a particular species, tissue, cell type, etc.; gene identification; etc.

Additional diagnostic uses for oligonucleotides designed from the sequences encoding a desired gene expression product may involve the use of PCR. These oligomers may be chemically synthesized, generated enzymatically, or produced in vitro. Oligomers will preferably contain a fragment of a polynucleotide encoding the expression products, or a fragment of a polynucleotide complementary to the polynucleotides, and will be employed under optimized conditions for identification of a specific gene. Oligomers may also be employed under less stringent conditions for detection or quantitation of closely-related DNA or RNA sequences.

### Pharmaceutical Compositions

The invention also includes pharmaceutical compositions containing nucleic acid conjugates. In some embodiments, the compositions are suitable for internal use and include an effective amount of a pharmacologically active conjugate of the invention, alone or in combination, with one or more pharmaceutically acceptable carriers. The conjugates are especially useful in that they have very low, if any toxicity.

The patient having a pathology, e.g. the patient treated by the methods of this invention can be a mammal, or more particularly, a human. In practice, the aptamers, are administered in amounts which will be sufficient to exert their desired biological activity.

The pharmaceutical compositions of the invention may contain, for example, more than one aptamer specificity. In some examples, a pharmaceutical composition of the invention, containing one or more compounds of the invention, is administered in combination with another useful composition such as an anti-inflammatory agent, an immunostimulator, a chemotherapeutic agent, an antiviral agent, or the like. Furthermore, the compositions of the invention may be administered in combination with a cytotoxic, cytostatic, or chemotherapeutic agent such as an alkylating agent, anti-metabolite, mitotic inhibitor or cytotoxic antibiotic, as described above. In general, the currently available dosage forms of the known therapeutic agents for use in such combinations will be suitable.

Combination therapy (or “co-therapy”) includes the administration of an aptamer compsoition and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected).

Combination therapy may, but generally is not, intended to encompass the administration of two or more of these therapeutic agents as part of separate monotherapy regimens that incidentally and arbitrarily result in the combinations of the present invention. Combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents.

Sequential or substantially simultaneous administration of each therapeutic agent can be effected by any appropriate route including, but not limited to, topical routes, oral routes, intravenous routes, intramuscular routes, and direct absorption through mucous membrane tissues. The therapeutic agents can be administered by the same route or by different routes. For example, a first therapeutic agent of the combination selected may be administered by injection while the other therapeutic agents of the combination may be administered topically.

Alternatively, for example, all therapeutic agents may be administered topically or all therapeutic agents may be administered by injection. The sequence in which the therapeutic agents are administered is not narrowly critical sunless noted otherwise. Combination therapy also can embrace the administration of the therapeutic agents as described above in further combination with other biologically active ingredients. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

Therapeutic or pharmacological compositions of the present invention will generally comprise an effective amount of the active component(s) of the therapy, dissolved or dispersed in a pharmaceutically acceptable medium. Pharmaceutically acceptable media or carriers include any and all solvents, dispersion media, coatings, antibacterial and antifungal agents, isotonic and absorption delaying agents and the like. The use of such media and agents for pharmaceutical active substances is well known in the art. Supplementary active ingredients can also be incorporated into the therapeutic compositions of the present invention.

For any aptamer used in the methods of the invention, the therapeutically effective amount or dose can be estimated initially from activity assays in cell cultures and/or animals. For example, a dose can be formulated in animal models to achieve a circulating concentration range that includes the IC₅₀ as determined by activity assays (e.g., the concentration of the test compound, which achieves a half-maximal inhibition of the proliferation activity). Such information can be used to more accurately determine useful doses in humans.

Toxicity and therapeutic efficacy of the peptides described herein can be determined by standard pharmaceutical procedures in experimental animals, e.g., by determining the IC₅₀ and the LD₅₀ (lethal dose causing death in 50% of the tested animals) for a subject compound. The data obtained from these activity assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1). Dosage amount and interval may be adjusted individually to provide plasma levels of the active moiety which are sufficient to maintain therapeutic effects, termed the minimal effective concentration (MEC). The MEC will vary for each preparation, but can be estimated from in vitro and/or in vivo data, e.g., the concentration necessary to achieve 50-90% inhibition of a proliferation of certain cells may be ascertained using the assays described herein. Dosages necessary to achieve the MEC will depend on individual characteristics and route of administration. HPLC assays or bioassays can be used to determine plasma concentrations. Dosage intervals can also be determined using the MEC value. Preparations should be administered using a regimen, which maintains plasma levels above the MEC for 10-90% of the time, preferable between 30-90% and most preferably 50-90%. Depending on the severity and responsiveness of the condition to be treated, dosing can also be a single administration of a slow release composition described hereinabove, with course of treatment lasting from several days to several weeks or until cure is effected or diminution of the disease state is achieved. The amount of a composition to be administered will, of course, be dependent on the subject being treated, the severity of the affliction, the manner of administration, the judgment of the prescribing physician, etc.

The preparation of pharmaceutical or pharmacological compositions will be known to those of skill in the art in light of the present disclosure. Typically, such compositions may be prepared as injectables, either as liquid solutions or suspensions; solid forms suitable for solution in, or suspension in, liquid prior to injection; as tablets or other solids for oral administration; as time release capsules; or in any other form currently used, including eye drops, creams, lotions, salves, inhalants and the like. The use of sterile formulations, such as saline-based washes, by surgeons, physicians or health care workers to treat a particular area in the operating field may also be particularly useful. Compositions may also be delivered via microdevice, microparticle or other known methods.

Upon formulation, therapeutics will be administered in a manner compatible with the dosage formulation, and in such amount as is pharmacologically effective. The formulations are easily administered in a variety of dosage forms, such as the type of injectable solutions described above, but drug release capsules and the like can also be employed.

In this context, the quantity of active ingredient and volume of composition to be administered depends on the host animal to be treated. Precise amounts of active compound required for administration depend on the judgment of the practitioner and are peculiar to each individual.

A minimal volume of a composition required to disperse the active compounds is typically utilized. Suitable regimes for administration are also variable, but would be typified by initially administering the compound and monitoring the results and then giving further controlled doses at further intervals.

For instance, for oral administration in the form of a tablet or capsule (e.g., a gelatin capsule), the active drug component can be combined with an oral, non-toxic, pharmaceutically acceptable inert carrier such as ethanol, glycerol, water and the like. Moreover, when desired or necessary, suitable binders, lubricants, disintegrating agents, and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes, and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol, and the like. Disintegrators include, without limitation, starch, methyl cellulose, agar, bentonite, xanthan gum starches, agar, alginic acid or its sodium salt, or effervescent mixtures, and the like. Diluents, include, e.g., lactose, dextrose, sucrose, mannitol, sorbitol, cellulose and/or glycine.

The compositions of the invention can also be administered in such oral dosage forms as timed release and sustained release tablets or capsules, pills, powders, granules, elixirs, tinctures, suspensions, syrups and emulsions. Suppositories are advantageously prepared from fatty emulsions or suspensions.

The pharmaceutical compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating, or coating methods, and typically contain about 0.1% to 75%, preferably about 1% to 50%, of the active ingredient.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in liquid prior to injection can be formulated.

The compositions of the present invention can be administered in intravenous (both bolus and infusion), intraperitoneal, subcutaneous or intramuscular form, all using forms well known to those of ordinary skill in the pharmaceutical arts. Injectables can be prepared in conventional forms, either as liquid solutions or suspensions.

Parenteral injectable administration is generally used for subcutaneous, intramuscular or intravenous injections and infusions. Additionally, one approach for parenteral administration employs the implantation of a slow-release or sustained-released systems, which assures that a constant level of dosage is maintained, according to U.S. Pat. No. 3,710,795, incorporated herein by reference.

Furthermore, preferred compositions for the present invention can be administered in intranasal form via topical use of suitable intranasal vehicles, inhalants, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would typically range from 0.01% to 15%, w/w or w/v.

For solid compositions, excipients include pharmaceutical grades of mannitol, lactose, starch, magnesium stearate, sodium saccharin, talcum, cellulose, glucose, sucrose, magnesium carbonate, and the like. The active compound defined above, may be also formulated as suppositories, using for example, polyalkylene glycols, for example, propylene glycol, as the carrier. In some embodiments, suppositories are advantageously prepared from fatty emulsions or suspensions.

The compounds of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564. For example, the aptamer molecules described herein can be provided as a complex with a lipophilic compound or non-immunogenic, high molecular weight compound constructed using methods known in the art. An example of nucleic-acid associated complexes is provided in U.S. Pat. No. 6,011,020.

The compounds of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

If desired, the pharmaceutical composition to be administered may also contain minor amounts of non-toxic auxiliary substances such as wetting or emulsifying agents, pH buffering agents, and other substances such as for example, sodium acetate, and triethanolamine oleate. The dosage regimen utilizing the aptamer is selected in accordance with a variety of factors including type, species, age, weight, sex and medical condition of the patient; the severity of the condition to be treated; the route of administration; the renal and hepatic function of the patient; and the particular aptamer or salt thereof employed. An ordinarily skilled physician or veterinarian can readily determine and prescribe the effective amount of the drug required to prevent, counter or arrest the progress of the condition.

Oral dosages of the present invention, when used for the indicated effects, will range between about 0.05 to 7500 mg/day orally. The compositions are preferably provided in the form of scored tablets containing 0.5, 1.0, 2.5, 5.0, 10.0, 15.0, 25.0, 50.0, 100.0, 250.0, 500.0 and 1000.0 mg of active ingredient. Infused dosages, intranasal dosages and transdermal dosages will range between 0.05 to 7500 mg/day. Subcutaneous, intravenous and intraperitoneal dosages will range between 0.05 to 3800 mg/day. Effective plasma levels of the compounds of the present invention range from 0.002 mg/mL to 50 mg/mL. Compounds of the present invention may be administered in a single daily dose, or the total daily dosage may be administered in divided doses of two, three or four times daily.

### Kits

In yet another aspect, the invention provides kits with desired aptamers.

In one embodiment, a kit comprises: (a) an aptamer that targets a desired cell and an immune cell stimulatory and/or co-stimulatory molecule, and (b) instructions to administer to cells or an individual a therapeutically effective amount of aptamer. In some embodiments, the kit may comprise pharmaceutically acceptable salts or solutions for administering the aptamer. Optionally, the kit can further comprise instructions for suitable operational parameters in the form of a label or a separate insert. For example, the kit may have standard instructions informing a physician or laboratory technician to prepare a dose of aptamer.

Optionally, the kit may further comprise a standard or control information so that a patient sample can be compared with the control information standard to determine if the test amount of an aptamer is a therapeutic amount consistent with for example, a shrinking of a tumor or decrease in viral load in a patient.

The invention has been described in detail with reference to preferred embodiments thereof. However, it will be appreciated that those skilled in the art, upon consideration of this disclosure, may make modifications and improvements within the spirit and scope of the invention. The following non-limiting examples are illustrative of the invention.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

Embodiments of the invention may be practiced without the theoretical aspects presented. Moreover, the theoretical aspects are presented with the understanding that Applicants do not seek to be bound by the theory presented.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

### Example 1

### PSMA-4-1BB Bispecific Aptamer

The PSMA-4-1BB aptamer is composed of a bivalent 4-1BB aptamer which binds and costimulates CD8⁺ T cells conjugated to a human PSMA binding aptamer. The PSMA aptamer targets the 4-1BB aptamer to PSMA expressing tumor cells in vivo and the 4-1BB bivalent aptamer costimulates the tumor infiltrating T cells.

In Vitro Functional Characterization—Binding to PSMA Expressing Cells and CD8+ T Cell Costimulation. To evaluate the binding of the bispecific aptamer to human PSMA expressing tumor cells, murine CT26 tumor cells were stably transfected with PSMA-expressing plasmids and binding of Cy3-labeled bispecific aptamer was monitored by confocal microscopy. A wild type and mutant human PSMA plasmid were stably transfected into murine CT26 tumor cells. The mutation consisted of a short deletion in the cytoplasmic domain which abolished PSMA internalization upon ligand binding. Binding of Cy3-labeled PSMA-4-1BB aptamer was monitored by confocal microscopy. The results showed that the PSMA-4-1BB aptamers bind to PSMA-expressing, but not nontransfected, tumor cells. Using wild type and mutant PSMA expressing cells, the bound aptamers internalize or remain on the cell surface, respectively. In the latter instance, the cells become essentially “coated” with the bispecific aptamers, presumably displaying the 4-1BB aptamer on the cell surface free to interact with tumor infiltrating CD8⁺ T cells.

To determine if the PSMA conjugated 4-1BB dimeric aptamer form retains its function, the costimulatory capacity of the PSMA conjugated 4-1BB aptamer was evaluated and compared to that of free 4-1BB aptamer dimer and 4-1BB Antibody. To show the costimulation of CD8⁺ T cells by the bispecific PSMA-4-1BB aptamer, bispecific aptamer, 4-1BB aptamer and 4-1BB antibody were incubated with CFSE-labeled polyclonally activated CD8⁺ T cells. Proliferation was measured by flow cytometry. The results showed that the bispecific aptamer was able to costimulate CD8⁺ T cells in vitro which was comparable to that of free 4-1BB aptamer or 4-1BB antibody.

Inhibition of Tumor Growth in Mice Treated with the Bispecific PSMA-4-1BB Aptamers:

Mice were implanted subcutaneously with ΔPSMA-expressing CT26 tumor cells and 3 days later injected with aptamer-siRNA chimeras. A partial and transient inhibition of tumor growth by the PSMA conjugated mutant 4-1BB aptamer may be attributed to nonspecific immune stimulation by nucleic acids. The results showed that tumor growth was significantly inhibited when day 3 old subcutaneously implanted tumor bearing mice were treated with bispecific PSMA-4-1BB aptamers. Therapy with a bispecific aptamer bearing a nonfunctional 4-1BB Moiety Exhibited a Partial and Transient Inhibition of Tumor Growth Most Likely Reflecting the Nonspecific Immune Stimulatory Effects of Nucleic Acids.

To demonstrate that the observed inhibition was a PSMA-targeted localized antitumor response, mice were co-implanted with ΔPSMA-expressing and non-transfected tumor cells and treated with the aptamer-siRNA chimeras. Mice were implanted with wild type CT26 and ΔPSMA-expressing CT-26 tumor cells in opposite flanks and treated with unconjugated 4-1BB aptamers, PSMA aptamer conjugated to a mutant 4-1BB aptamer or to PSMA conjugated to a functional 4-1BB aptamer. Mice were sacrificed when the nontransfected CT26 tumors reached 1.2 cm diameter. The results further showed that mice treated with the bispecific PSMA-4-1BB aptamer rejected the PSMA-expressing but not the non-transfected tumor cells.

Summary:

A bispecific PSMA-4-1BB aptamer homes to PSMA-expressing tumor cells in vivo to promote local 4-1BB mediated costimulation and tumor rejection.

The PSMA-4-1BB aptamer is a first prototype of a new class of oligonucleotide-based bispecific agents that can be used to deliver therapeutic aptamers to specific cells in vivo, or to attract cells to each other such as cytotoxic T cells to tumor cells.

The oligonucleotide nature of bispecific aptamers which unlike antibodies can be synthesized in cell-free chemical reaction, offers a simpler and cost effective way to develop clinical grade reagents to test and use in human therapy.

### Example 2

### Targeting 4-1BB Costimulation to Disseminated Tumor Lesions with Bi-Specific oligonucleotide aptamers

The development of bi-specific ligands composed of oligonucleotide (ODN) aptamers (Gold, L. 1995. J Biol Chem 270:13581-13584; Nimjee, S. M., C. P. Rusconi, and B. A. Sullenger. 2005. Annu Rev Med 56:555-583) to target costimulatory ligands to tumor cells in vivo is described. One aptamer, the therapeutic aptamer, which binds to and activates a costimulatory receptor, is conjugated to a second aptamer, the targeting aptamer, which binds to a tumor-specific product expressed on the cell surface and targets the therapeutic aptamer to tumor lesions in vivo. Unlike protein or monoclonal antibody reagents, the short ODN-based aptamers can be synthesized in a cell-free cost-effective chemical process, and exhibit little to no immunogenicity upon repeated administrations in vivo. In this study an agonistic 4-1BB binding aptamer conjugated to a PSMA-binding aptamer was targeted to PSMA-expressing tumors in mice and was shown to inhibit tumor growth.

Materials and Methods:

PSMA-4-1BB Aptamer Conjugates:

The PSMA aptamer, 5′GGGAGGACGAUGCGGAUCAGCCAUGUUUACGUCACUCCUUGUCAAUCCUCAUC GGCAGACGACUCGCCCGA 3′ (SEQ ID NO: 1) was cloned into pUC57 between KpnI and BamHI restriction sites and PCR amplified using forward primer 5′TAATACGACTCACTATAGGGAGGACGATGCGG3′ (SEQ ID NO: 2) and reverse primer 5′GCTATAAGTGTGCATGAGAACTCGGGCGAGTCGTCTG3′ (SEQ ID NO: 3). The reverse primer encodes a sequence which is complementary to the linker sequence of the dimeric form of 4-1BB aptamer. Using overlapping oligonucleotides the 4-1BB dimer was cloned in pGem-t-easy plasmid (Promega, Madison, Wis.) and PCR amplified using the forward primer 5′CAGGCGGCCGCGAATT3′ (SEQ ID NO: 4) and reverse primer 5′CGTCGCATGCTCCCGGC3′ (SEQ ID NO: 5). To generate a mutated form of the 4-1BB dimer, (mut4-1BB), PCR amplification was carried out in the presence of 8-oxo-2′-deoxyguanosine-5′-Triphosphate and 2′-deoxy-pyrene-5′-triphosphate (Trilink, Millersvile, Md.). The PCR products were cloned into pGem-t-easy plasmid and sequenced. A clone that had no predicted impact in the secondary structure of the PSMA aptamer was chosen for further studies.

The sequence of the 4-IBB dimer:
(SEQ ID NO: 6)
5′GGGAGAGAGGAAGAGGGAUGGGCGACCGAACGUGCCCUUCAAAGCCG
UUCACUAACCAGUGGCAUAACCCAGAGGUCGAUAGUACUGGAUCCCCCC
CCCGCUAUAAGUGUGCAUGAGAACCCCGGGGGGAGAGAGGAAGAGGGAU
GGGCGACCGAACGUGCCCUUCAAAGCCGUUCACUAACCAGUGGCAUAAC
CCAGAGGUCGAUAGUACUGGAUCCCCCC3′.
The sequence of mut4-IBB dimer:
(SEQ ID NO: 7)
5′GGGAGAGAGGAAGGGGGAUGGGCGACCGAGCGUGCCCUCCAGAGCCG
UUCACCAGCCAGUGGCAUAGCCCAGAGGUCGAUAAUACUGGACCCCCCC
CCCGCUAUAAGCGGGCAUGAGAACCCCGGGGGGAGAGAGGAAGGGGGAU
GGGCGACCGAACGUGCCCCUCAAAGCCGUCCACUAACCAGCGGCACAGC
CCAGAGGCCGAUAGUACUGGACCCCCCC3′.

The PSMA and 4-1BB aptamer PCR products were purified using the QIAprep Spin columns (Qiagen, Valencia, Calif.). RNA was transcribed using the T7(Y639F) polymerase as previously described (60) and annealed to form the PSMA-4-1BB aptamer conjugates. The products were separated on a polyacrylamide gel. The conjugate was purified by polyacrylamide gel electrophoresis and concentrated on a 30 Kda Amicon Ultra-4 column (Millipore, Billerica, Mass.).

Confocal Microscopy:

The 4-1BB aptamer dimer was labeled with Cy3 before hybridization to the PSMA aptamer using the Silencer RNA labeling kit (Ambion, Austin, Tex.). Tumor cells were washed with PBS and incubated with 40 nM of Cy3-labeled aptamer conjugate or with 10 mg/ml anti-PSMA Ab (MBL, Woburn, Mass.) and Alexa Fluor 488 goat anti-mouse IgG (Molecular Pobes, Eugene, Oreg.). Coverslips were mounted with Prolong Gold-DAPI (Molecular Probes, Eugene, Oreg.).

Derivation of PSMA-expressing CT26 tumor cell lines: The PSMA cDNA was
PCR amplified using forward primer
(SEQ ID NO: 8)
5′GATCAGCGGCCGCGCCACCATGTGGAATCTCCTTCACG3′
and
reverse primer
(SEQ ID NO: 9)
5′GTTAAGTCGACGAGGATCCTCGAGAATCCTCTTAGGCTACTTCACTC3′.
ΔPSMA was generated by deleting the N-terminal 13 amino acids
(SEQ ID NO: 10)
WNLLHETDSAVAT
using forward primers
(SEQ ID NO: 11)
5′GATCAGCGGCCGCGCCACCATGGCGCGCCGCCCGCGCTGGCTG3′
and
reverse primer
(SEQ ID NO: 12)
5′GTTAAGTCGACGAGGATCCTCGAGAATCCTCTTAGGCTACTTCACTC3′.

The PCR products were cloned into the SalI and Not1 restriction sites of the retroviral vector pBMN (Addgene, Cambridge, Mass.) and transiently transfected into the Phoenix-AMPHO 293 packaging cell lines. Viral supernatant was used to transduce CT26 colon carcinoma (H-2^{d}) and B16/F10 melanoma (H-2^{b}) tumor cell lines and PSMA-expressing cells were isolated by sorting using PE-labeled anti-PSMA antibody from MBL, Woburn, Mass.

CFSE Proliferation:

CD8 cells were isolated from spleen and lymph nodes, using the stem Sep negative selection kit (StemCell Technologies, Vancouver, Calif.). Purified CD8 cells were stained with 2 μM of CFSE. CFSE labeled cells were incubated with 1 mg/ml of CD3 (BD Bioscience, San Jose, Calif.) and either 5 mg/ml of anti-murine 4-1BB (3H3) Ab kindly provided by Dr. Robert Mittler, 5 mg/ml isotype control Ab, 100 nM of 41BB aptamer dimer or 100 nM PSMA-4-1BB aptamer conjugates and analyzed by flow cytometry.

Intraturmoral Infiltration of Pmel-1 CD8+ T Cells:

C57BL/6 mice (Thy 1.2) were implanted subcutaneously in one flank with 3×10⁴ B16/F10 tumor cells and in the contralateral flank with 10⁵ ΔPSMA-B16/F10 tumor cells. At day 6, 5×10⁶ gp100-specific Pmel-1 (Thy 1.1) CD8⁺ T cells were injected via the tail vein and two days later injected with 5 mg of gp100 peptide (KVPRNEDWL (SEQ ID NO: 13)) plus 5 mg LPS. At days 11, 12, 13 and 17 mice were injected with 50 pmoles of PSMA-4-1BB aptamer conjugate. In some mice 100 pmoles of 4-1BB-Ig fusion protein or isotype IgG (R&D, Minneapolis, Minn.) were injected intratumorally coincident with injection of aptamer conjugates. Mice were sacrificed at day 21, tumors were removed, treated with collagenase, stained with APC-labeled anti-CD8 Ab (BD Bioscience, San Jose, Calif.) and anti PE-labeled anti-Thy1.1 Ab (BD Bioscience, San Jose, Calif.) and analyzed by flow cytometry.

Tumor Immunotherapy Studies:

3×10⁵ parental CT26 or ΔPSMA-CT26 tumor cells were implanted subcutaneously in Balb/c mice. At days 3, 4, 5 and 7 the tumor-bearing mice were injected via the tail vein with 50 pmoles of PSMA-4-1BB, 50 or 500 pmoles of 4-1BB aptamer dimer, or with 50 or 500 pmoles of anti-4-1BB 3H3 antibody.

To monitor for metastasis, C57BL/6 mice were implanted with 10⁵ ΔPSMA-B16/F10 cells via the tail vein and injected with 50 pmoles PSMA-4-1BB aptamer conjugates at days 5, 8, 11, 14. When about half of the mice in the control groups have shown signs of morbidity (circa days 25-28), mice were sacrificed and lungs were weighed. GM-CSF expressing B16/F10 tumor cells, were irradiated (5000 rad) and 5×10⁵ cells were injected subcutaneously at days 5, 8 and 11.

Statistical Analysis:

For statistical analysis of tumor growth P values were calculated using Student's t-test. For survival, P values were determined using the Log-rank (Mantel-Cox) test.

Tumor Homing or ³²P-Labeled Aptamer Conjugates.

The PSMA aptamer was transcribed in vitro in the presence of 1/1000 parts of α³²P-ATP (3000Ci/mmol) (PerkinElmer, Boston Mass.) and annealed to 4-1BB aptamer as described above. Balb/c mice were co-implanted with CT26 and PSMA-CT26 tumor cells in the opposite flanks and 15 days later injected via the tail vein with 5×10⁵ cpm ³²P-labeled aptamer conjugate. At indicated times tumors were surgically removed, cells dispersed by incubation with 400 U/ml of collagenase, washed three times with PBS, and cell associated ³²P was measured in a scintillation counter.

Results:

Functional Characterization of a Bi-Specific PSMA-4-1BB Aptamer Conjugate:

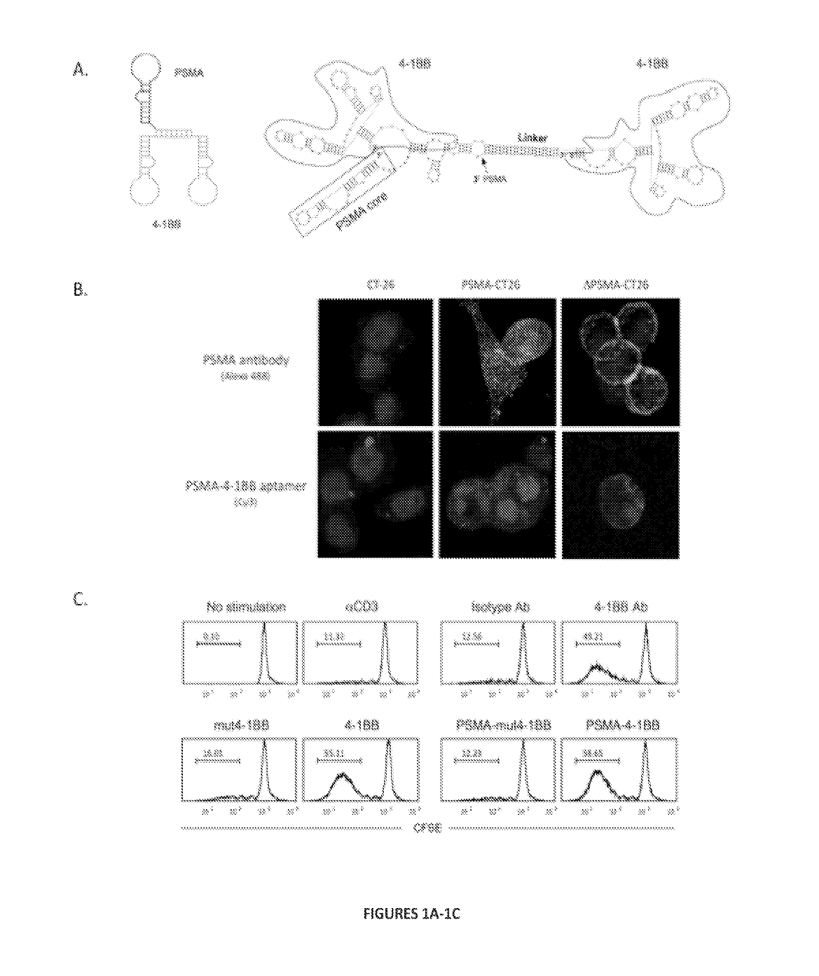

4-1BB is a major costimulatory receptor promoting the survival and expansion of activated CD8⁺ T cells. A bivalent 4-1BB binding aptamer formed by conjugation of two monomeric aptamers costimulatesd CD8′ T cells and promoted tumor immunity in mice. To target the agonistic 4-1BB aptamer to tumor cells, the bivalent dimeric aptamer (“4-1BB aptamer”) was conjugated to a PSMA-binding aptamer as shown in FIG. 1A. PSMA is a prostate tissue specific product which is upregulated on human prostate tumor cells. To analyze the immunological and antitumor effects of PSMA-targeted costimulation in immune competent mice, murine CT26 colorectal carcinoma (H-2^{d}) and B16/F10 melanoma (H-2^{b}) tumor cells were stably transfected with a human PSMA expression plasmid.

Tumor targeted 4-1BB costimulatory ligands, like cytotoxic antibodies such as RITUXIMAB or TRASTUZUMAB that kill their tumor target via ADCC or fix complement, need to engage receptors that either do not internalize or rapidly recycle without dissociating their cargo. Binding of RITUXIMAB to CD20 or m816C antibody to TENASCIN (are examples of the former, while binding of TRASTUZUMAB to Erb2 may be an example of the latter, though Erb2 may not internalize efficiently upon antibody binding. PSMA, like many receptors, upon binding its ligand is internalized via a clathrin-dependent endocytic mechanism. Thus, in order to simulate a non internalizing receptor, a deletion was introduced into the cytoplasmic domain of PSMA (ΔPSMA) that was shown to reduce its internalization upon ligand binding.

Both PSMA and ΔPSMA, were stably transfected into CT26 colon carcinoma and B16/F10 melanoma tumor cells. Flow cytometry confirmed that both PSMA proteins are expressed at comparable levels on the surface of transfected tumor cells. The binding and subcellular localization of the PSMA-4-1BB aptamer conjugate in CT26 tumor cells was determined by confocal microscopy. As shown in FIG. 1B, the PSMA-4-1BB aptamer conjugates or anti-PSMA antibody bind to PSMA-CT26 cells and are internalized, whereas they bind to ΔPSMA-CT26 cells but remain on the cell surface. It was next tested whether the 4-1BB aptamer when conjugated to the PSMA aptamer retained its capacity to costimulate CD8 T cells. Costimulation was determined by measuring the proliferation of suboptimally activated CFSE-labeled CD8⁺ T cells. As shown in FIG. 1C, 4-1BB antibody, unconjugated 4-1BB aptamer, and the PSMA-conjugated 4-1BB aptamer, but not PSMA-mut4-1BB which contains a nonfunctional 4-1BB aptamer, induced a comparable level of T cell proliferation. These experiments have shown that conjugation of the PSMA and 4-1BB aptamer has not adversely affected their respective functions, binding to PSMA-expressing cells and costimulation, respectively.

Inhibition of Tumor Growth in Mice Treated with PSMA-4-1BB Aptamer Conjugates.

It was next determined whether systemic administration of PSMA-4-1BB aptamer conjugates can impact on tumor growth in tumor-bearing mice using the poorly immunogenic subcutaneously implanted CT26 colon carcinoma and the B16 clone F10 (B16/F10) lung metastasis models. Dose titration experiments have shown no significant differences in the growth potential of parental and ΔPSMA-expressing CT26 or B16/F10 tumor cells. Treatment of day 3 subcutaneously implanted ΔPSMA-CT26 tumor-bearing mice with PSMA-4-1BB aptamer conjugate had a profound inhibitory effect on tumor growth (FIG. 2A), 4 out of 10 mice surviving long-term (FIG. 2B). The mice which rejected the implanted tumor shown in FIG. 2B, but not age-matched control mice, were resistant to a rechallenge with CT26 tumor cells. This observation evidences that tumor targeted 4-1BB costimulation, in addition to inducing the rejection of the targeted tumor, also engenders long-term protective immunological memory against the parental tumors not targeted with 4-1BB aptamer ligands. It was noted repeatedly that injection of PSMA aptamer conjugated to non-functional “cargo” such as control siRNAs or mutant 4-1BB as shown in FIG. 2A or FIG. 2B, had a small inhibitory effect on tumor growth, seen in some but not all experiments. This can be attributed either to nonspecific immune stimulation by the ODN backbone of the aptamer conjugate or a result of direct binding to the PSMA-expressing tumor cells. In a second model the ability of the PSMA-4-1BB aptamers to inhibit lung metastasis was evaluated. To this end, C57BL/6 mice were injected intravenously with ΔPSMA-B16/F10 tumor cells and treated with PSMA-4-1BB or PSMA-mut4-1BB aptamer conjugates starting at day 5 post tumor inoculation. As shown in FIG. 2C, PSMA-4-1BB, but not PSMA-mut4-1BB treatment inhibited the development of lung metastasis. By visual inspection at the time of sacrifice, 6 out of 8 mice in the PSMA-4-1BB treatment groups were free of metastasis whereas the lungs of all mice from the control and PSMA-mut4-1BB treated groups were covered with many metastatic nodules.

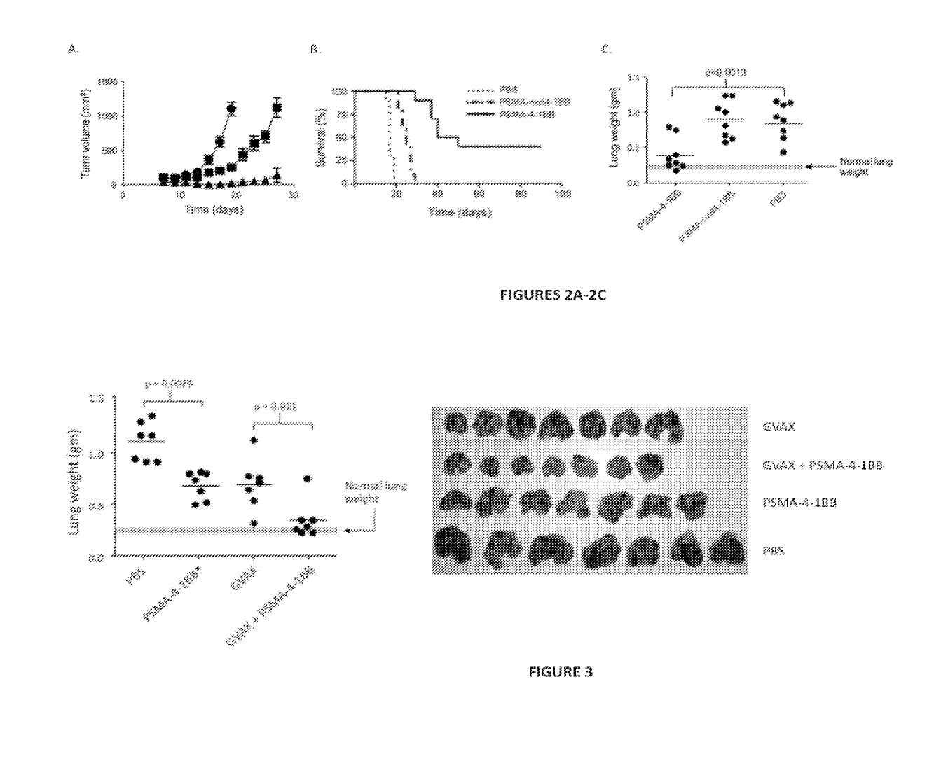

It was tested next whether tumor-targeted costimulation can potentiate a vaccine-induced antitumor response. To this end, day 5 B16/F10 tumor bearing mice were vaccinated with GM-CSF expressing irradiated B16/F10 tumor cells (GVAX) and/or treated with PSMA-4-1BB aptamer conjugates. GVAX vaccination of tumor bearing mice resulted in a partial inhibition of metastasis (FIG. 3), thereby simulating a “weak” vaccination protocol. To measure synergy between vaccination and costimulation, the PSMA-4-1BB aptamer conjugates were injected at half the concentration used in FIG. 2C to prevent the almost complete inhibition of metastasis by this treatment alone. As shown in FIG. 3, GVAX vaccination combined with 4-1BB costimulation was significantly more effective compared to each treatment alone. Interestingly, 3 out 7 mice in this group developed coat discoloration reminiscent of vitilago, an antimelanocyte autoimmune response (FIG. 7).

Mechanism of Tumor Inhibition—PSMA Targeting and 4-1BB Costimulation.

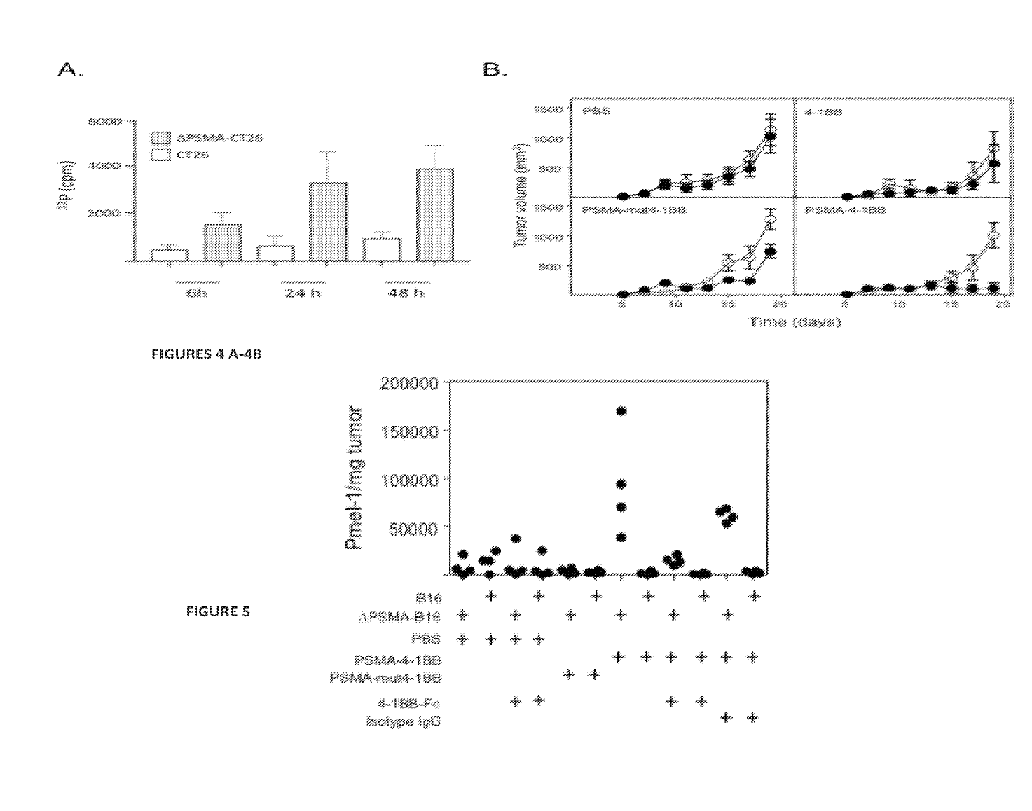

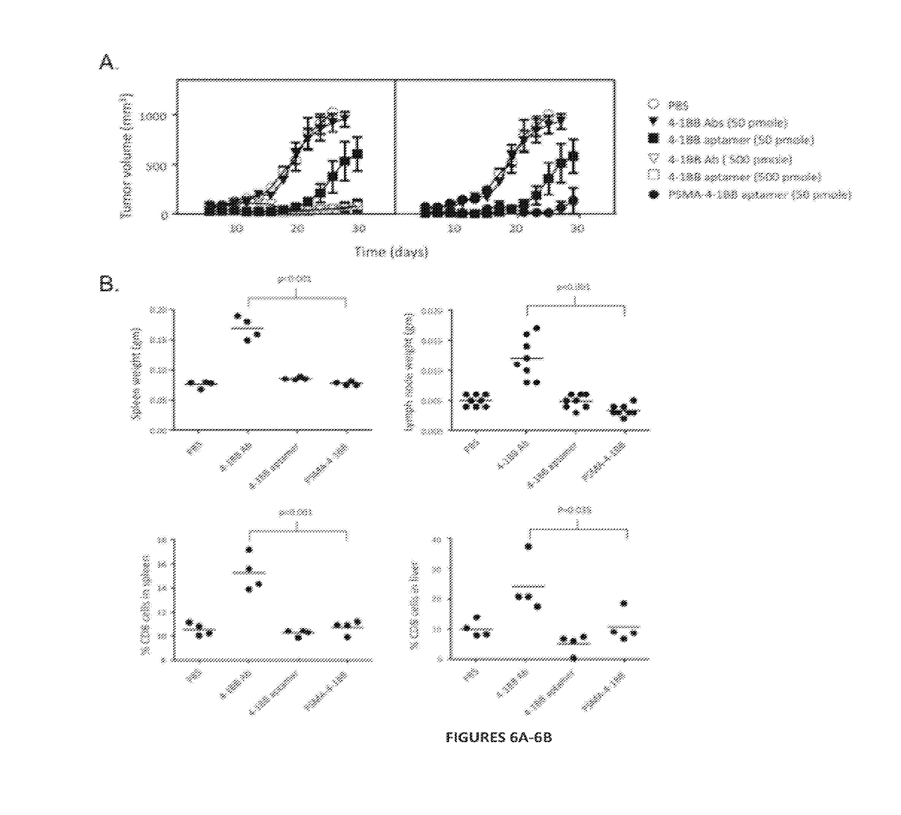

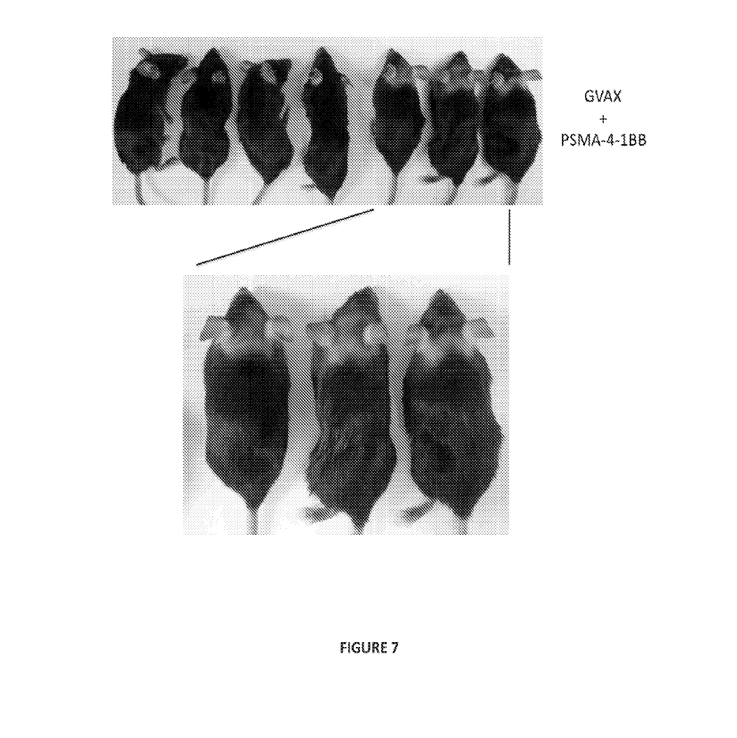

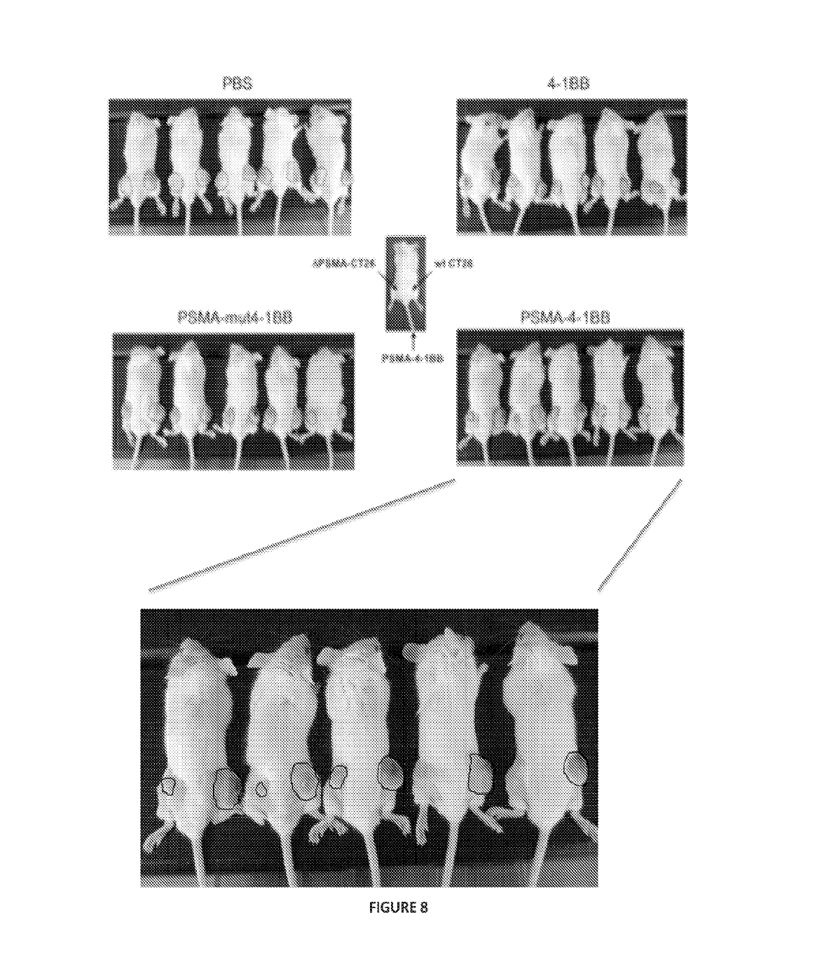

To determine whether tumor inhibition seen in FIGS. 2A-2C is dependent on PSMA targeting, mice were co-implanted in opposite flanks with parental CT26 and ΔPSMA-CT26 tumor cells and injected via the tail vein with either PSMA-4-1BB aptamer conjugate, unconjugated 4-1BB aptamer, or the costimulatory-deficient PSMA-mut4-1BB aptamer conjugate. FIG. 4A, shows that the systemically injected ³²P-labeled PSMA-4-1BB aptamer conjugates accumulated preferentially in the ΔPSMA-expressing as compared to parental CT26 tumor cells. FIG. 4B shows that treatment with the PSMA-4-1BB aptamer conjugate inhibited the growth of PSMA-expressing, but not the contralaterally implanted parental CT26 tumor cells. Treatment with unconjugated 4-1BB had a small inhibitory effect on both ΔPSMA-expressing and parental tumor cells reflecting the effect of limited 4-1BB costimulation at the concentration used (see also FIG. 6A below). Treatment with the costimulatory-deficient PSMA-mut4-1BB aptamer conjugate had a small inhibitory effect on the ΔPSMA-expressing, but not the contralaterally implanted parental tumor cells. This is consistent with the experiment shown in FIG. 2A and implies that the observed inhibition reflects binding of aptamer conjugates to the (PSMA expressing) tumor cells rather than a nonspecific immune stimulatory effect of nucleic acids. FIG. 8 shows that at day 19, when mice were sacrificed because the parental CT26 tumors reached maximum allowable size, only PSMA-expressing, but not parental, CT26 tumors in mice treated with PSMA-4-1BB aptamer conjugate exhibited significant inhibition of growth; in three mice small tumors were palpable whereas in two mice tumors initially grew, became palpable, but fully regressed at the time of sacrifice.

This experiment, therefore, shows that the inhibition of tumor growth is mediated via PSMA aptamer targeting to tumor cells expressing the cognate receptor, it depends on a costimulation-competent 4-1BB aptamer, and that inhibition is, at least initially, local. This, however, appears to conflict with the repeated observations that mice which rejected the CT26 tumors, as shown in FIG. 2B or FIG. 6A below, were resistant to a subsequent tumor challenge, evidencing that aptamer treatment induced systemic, and not local, antitumor immunity. A plausible explanation that reconciles both observations is that the tumor-targeted 4-1BB costimulation does potentiate a systemic immune response but its dissemination is delayed, becoming effective if the tumor challenge occurs subsequent to PSMA-4-1BB treatment, but not if tumor “challenge” is concurrent with PSMA-4-1BB therapy.

To obtain direct evidence that the PSMA-4-1BB aptamer conjugate is capable of costimulating tumor infiltrating CD8⁺ T cells in a 4-1BB-dependent manner, it was determined whether intratumoral accumulation of tumor-specific CD8+ T cells is dependent on 4-1BB/4-1BBL interactions. C57BL/6 mice were implanted subcutaneously with parental or with ΔPSMA-expressing B16/F10 tumor cells and at day 6 transgenic Pmel-1 CD8⁺ T cells which recognize an epitope of gp100, a tumor antigen expressed in B16/F10 tumor cells, were injected via the tail vein. Starting at day 11 post tumor implantation, mice were treated with PSMA-4-1BB aptamer conjugate, PSMA-mut4-1BB aptamer conjugate or PBS by tail vein injection. At day 21 mice were sacrificed, tumor excised, and the intratumoral infiltration of Pmel-1 cells was determined by flow cytometry. Where indicated, mice were also treated intratumorally with 4-1BB-Fc fusion or with isotype IgG. The results of such an experiment is shown in FIG. 5. The only combination that resulted in significant intratumoral accumulation of Pmel-1 cells were mice implanted with a ΔPSMA-B16 tumor and treated with PSMA-4-1BB aptamer conjugate. Importantly, treatment with a 4-1BB-Fc fusion which blocks 4-1BB/4-1BBL interactions, but not isotype control, inhibited the intratumoral accumulation of the Pmel-1 cells. This experiment, therefore, shows that PSMA-4-1BB mediated intraturmoral accumulation of the B16 tumor-specific Pmel-1 CD8⁺ T cell was dependent on 4-1BB costimulation as well as PSMA targeting. In summary, the two experiments shown in FIGS. 4A, 4B and 5 provide complementary evidence that systemic administration of PSMA-4-1BB aptamer conjugates inhibits tumor growth in mice via tumor-targeted (PSMA aptamer-dependent) 4-1BB costimulation.

Tumor Targeting Improves the Safety Profile of 4-1BB Costimulation.

Since the systemic administration of agonistic 4-1BB antibodies results in nonspecific immune stimulation (Lee, S. W., et al. 2009. J Immunol 182:6753-6762; Niu, L., S. et al. 2007. J Immunol 178:4194-4213.), it was tested whether tumor-targeted delivery of the 4-1BB aptamer ligands will reduce the severity of adverse effects associated with 4-1BB costimulation. Using the subcutaneous CT26 tumor model described in FIG. 2A, the antitumor activity of an agonistic anti-4-1BB antibody, unconjugated 4-1BB aptamer, and the PSMA-4-1BB aptamer conjugate, were compared. The results of this experiment shown in FIG. 6A are displayed in two panels for clarity purposes. The left panel shows that systemic administration of 500 pmoles of unconjugated 4-1BB aptamer or 4-1BB antibody inhibits tumor growth almost completely. However, when the concentration of aptamer and antibody was reduced ten-fold to 50 pmoles, the antibody failed to inhibit tumor growth whereas the 4-1BB aptamer exerted a partial inhibitory effect. This is consistent with the immune modulatory CTLA-4, 4-1BB or OX40 aptamers which were equally or slightly more potent than the corresponding antibodies. The right panel shows that 50 pmole of the PSMA-targeted 4-1BB aptamer conjugate was as or almost as effective as 500 pmoles of 4-1BB aptamer or 4-1BB antibody in inhibiting tumor growth. This experiment, therefore, shows that targeting 4-1BB ligands to tumor cells will require less reagent than using untargeted ligand to achieve a therapeutic benefit, and that on a molar basis the PSMA-4-1BB aptamer conjugate was significantly superior to 4-1BB antibodies, the “gold standard” 4-1BB ligand used in clinical trials in cancer patients (clinicaltrials.gov).

In the experiment shown in FIG. 6B, the adverse effects were measured in mice treated with therapeutic doses of 4-1BB antibody (500 pmoles), unconjugated 4-1BB aptamer (500 pmoles) and PSMA conjugated 4-1BB aptamer (50 pmoles). Treatment of mice with 4-1BB antibody recapitulated the effects, which include enlarged spleen and lymph nodes and elevated levels of CD8⁺ T cells in the spleen and liver. In contrast, treatment of mice with unconjugated 4-1BB aptamer or with PSMA-4-1BB aptamer conjugate did not result in enlarged spleen or lymph nodes nor in the accumulation of CD8⁺ T cells in the spleen or liver. These results show that the unconjugated and PSMA-targeted 4-1BB aptamers exhibit a superior safety profile compared to antibodies, and that treatment with the tumor targeted 4-1BB aptamers requires 5-10-fold less reagent to achieve a therapeutic effect. It is tempting to speculate that the enhanced safety profile of unconjugated 4-1BB aptamer compared to 4-1BB antibody is due to its reduced plasma half-life of 6-18 hours compared to antibodies which can persist for one to three weeks.

Discussion:

In this study a novel, and potentially clinically useful, composition and method to promote costimulation at the site of disseminated tumors is described using bi-specific oligonucleotide aptamers to target costimulatory ligands to tumor cells in situ. Systemic administration of PSMA-4-1BB aptamer conjugates to tumor bearing mice led to significant inhibition of tumor growth and long-term tumor rejection (FIGS. 2A-2C, 4A, 4B and 6A, 6B). Moreover, targeted costimulation with bi-specific aptamers can synergize with and potentiate vaccine-induced immunity (FIG. 3). The magnitude of the protective antitumor response engendered by the PSMA-4-1BB bi-specific aptamers seen in this study (using a first generation reagent and non-optimized treatment schedule) has been rarely observed with other immune potentiating single-agent monotherapies and appears to be superior to that of vaccination with GM-CSF expressing irradiated tumor cells (GVAX), a best-in-class vaccination protocol in mice (FIGS. 2A-2C, 3). The tumor inhibitory effect of administering PSMA-4-1BB aptamer conjugates to the tumor bearing mice reflected the potentiation of a naturally occurring, though weak and nonproductive, immune response elicited by the poorly immunogenic CT26 or B16/F10 tumors. Since tumor progression in cancer patients often elicits, albeit ineffective, antitumor immune responses, these observations evidence that tumor-targeted costimulation may be capable of potentiating the naturally occurring antitumor immune responses in cancer patients and control tumor progression.

Drug toxicity, reflecting limited specificity of the drug to its target, is a major impediment in developing effective treatments for cancer. For example, in human volunteers, administration of superagonistic CD28 antibodies was associated with severe toxicity, and in mice administration of agonistic 4-1BB antibodies resulted in nonspecific immune stimulation and other immune-related anomalies. Targeting poorly specific drugs to tumor cells should mitigate their undesirable effects on normal cells. Here it was shown that using bi-specific aptamers to target 4-1BB ligands to tumor cells can reduce the therapeutic dose compared to untargeted ligand (FIG. 6A), and is not associated with adverse effects as compared to using a 4-1BB antibody (FIG. 6B). Thus, aptamer targeting of costimulatory aptamer ligands to tumor cells in vivo should increase their safety profile, i.e., their therapeutic index, as well as reduce the amount of reagent needed to achieve a therapeutic benefit.

Several studies have shown that optimal activation of T cells by costimulation thru 4-1BB(51-53), OX40(54, 55), or GITR(56-58) can promote their resistance to the immune suppressive effects of foxp3⁺ regulatory T cells (Treg) and conceivably other immune attenuating mediators. Effective costimulation targeted to the tumor site with bi-specific aptamers could, therefore, confer increased resistance to the local immune suppressive effects of Treg without affecting their physiological functions elsewhere in the body. It is, therefore, tempting to speculate, and which future studies will test, that effective tumor-targeted costimulation may reduce, the need to develop strategies to counter tumor-induced suppression mechanisms.

In summary, aptamer-based bi-specific ligands represent a new platform technology to endow costimulatory capacity to disseminated tumors which will synergize with vaccination protocols to enhance the susceptibility of disseminated tumors to naturally occurring or vaccine-induced antitumor immune responses. The PSMA-4-1BB aptamer conjugate described in this study is a first-generation prototype aptamer conjugate that can be used to deliver other, and perhaps more effective, costimulatory ligands to tumor cells such as CD70, CD40L or LIGHT. Targeted delivery of aptamer-based costimulatory ligands to tumor cells in situ could, therefore, be a powerful approach to engender protective antitumor immunity.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The Abstract of the Disclosure is provided to allow the reader to quickly ascertain the nature of the technical disclosure. It is submitted with the understanding that it will not be used to interpret or limit the scope or meaning of the following claims.

What is claimed:

1. A composition for tumor cell immunogenicity comprising an aptamer with specificity for at least one immune cell stimulatory molecule and one tumor antigen.

2. The composition of claim 1, wherein the aptamer is specific for a plurality of immune cell stimulatory molecules (multi-specificity).

3. The composition of claim 1, wherein the aptamer is specific for a plurality of tumor antigens (multi-specificity).

4. The composition of claim 1, wherein the aptamer is specific for a plurality of immune cell stimulatory molecules and at least one tumor antigen.

5. The composition of claim 1, wherein the aptamer is specific for at least one immune stimulatory molecule and a plurality of tumor antigens.

6. The composition of claim 1, wherein the composition comprises a plurality of aptamers with mono-specificity for an immune cell stimulatory and mono-specificity for a tumor antigen.

7. The composition of claim 1, wherein the aptamer is specific for immune cell stimulatory molecules comprising at least one of: 4-1BB (CD137), B7-1/2, 4-1BBL, OX40L, CD40, LIGHT, OX40, CD2, CD3, CD4, CD8a, CD11a, CD11b, CD11c, CD19, CD20, CD25 (IL-2Rα), CD26, CD27, CD28, CD40, CD44, CD54, CD56, CD62L (L-Selectin), CD69 (VEA), CD70, CD80 (B7.1), CD83, CD86 (B7.2), CD95 (Fas), CD134 (OX-40), CD137, CD137L, (Herpes Virus Entry Mediator (HVEM), TNFRSF14, ATAR, LIGHTR, TR2), CD150 (SLAM), CD152 (CTLA-4), CD 154, (CD40L), CD 178 (FasL), CD209 (DC-SIGN), CD 270, CD277, AITR, AITRL, B7-H3, B7-H4, BTLA, HLA-ABC, HLA-DR, ICOS, ICOSL (B7RP-1), NKG2D, PD-1 (CD279), PD-L1 (B7-H1), PD-L2 (B7-DC), TCR-α, TCR-β, TCR-γ, TCR-δ, ZAP-70, lymphotoxin receptor (LTβ), NK1.1, HLA-ABC, HLA-DR, T Cell receptor αβ (TCRαβ), T Cell receptor γδ (TCRγδ), T cell receptor ζ (TCRζ), TGFβRII, TNF receptor, Cd11c, CD1-339, B7, Foxp3, mannose receptor, or DEC205, variants, mutants, species variants, ligands, alleles and fragments thereof.

8. The composition of claim 1, wherein immune cells comprise T cells (T lymphocytes), B cells (B lymphocytes), antigen presenting cells, dendritic cells, monocytes, macrophages, myeloid suppressor cells, natural killer (NK) cells, NK T cells, suppressor cells, T regulatory cells (Tregs), cytotoxic T lymphocytes (CTLs), CTL lines, CTL clones, CTLs from tumor, inflammatory, or other infiltrates and subsets thereof.

9. The composition of claim 1, wherein the aptamer is specific for T lymphocyte stimulatory and/or co-stimulatory molecules.

10. The composition of claim 9, wherein the aptamer is specific for CD8⁺ T Lymphocyte stimulatory and/or co-stimulatory molecules.

11. The composition of claim 1, wherein the aptamer is specific for at least one tumor antigen comprising: PSMA; BRCA1, BRCA2, alpha-actinin-4; BCR-ABL fusion protein (b3a2); CASP-8; β-catenin; Cdc27; CDK4; dek-can fusion protein; Elongation factor 2; ETV6-AML1 fusion protein; LDLR-fucosyltransferase AS fusion protein; hsp70-2; KIAAO205; MART2; MUM-1f; MUM-2; MUM-3; neo-PAP; Myosin class I; OS-9g; pml-RAR alpha fusion protein; PTPRK; K-ras; N-ras; CEA; gp100/Pmel17; Kallikrein 4; mammaglobin-A; Melan-A/MART-1; PSA; TRP-1/gp75; TRP-2; tyrosinase; CPSF; EphA3; G250/MN/CAIX; HER-2/neu; Intestinal carboxyl esterase; alpha-fetoprotein; M-CSF; MUC1; p53; PRAME; RAGE-1; RU2AS; survivin; Telomerase; WT1; or CA125.

12. The composition of claim 1, wherein the tumor antigen is PSMA.

13. The composition of claim 1, wherein the at least one immune cell stimulatory binding aptamer is linked to the at least one tumor antigen binding aptamer by at least one linker molecule.

14. The composition of claim 13, wherein said linker molecule comprising: nucleotide, non-nucleotide, or mixed nucleotide/non-nucleotide molecules.

15. The composition of claim 13, wherein the one or more linker molecules comprising about 2 nucleotides length up to about 50 nucleotides in length.

16. The composition of claim 13, wherein the non-nucleotide linker comprises abasic nucleotide, polyether, polyamine, polyamide, peptide, carbohydrate, lipid, polyhydrocarbon, or polymeric compounds having one or more monomeric units.

17. The composition of claim 1, wherein the aptamer molecule comprises one or more nucleotide substitutions.

18. The composition of claim 17, wherein the nucleotide substitutions comprise at least one or combinations thereof, of adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynylcytosine, 5-fluorouracil, 5-bromouracil, pseudoisocytosine, 2-hydroxy-5-methyl-4-triazolopyridin, isocytosine, isoguanin, inosine, non-naturally occurring nucleobases, locked nucleic acids (LNA), peptide nucleic acids (PNA), variants, mutants and analogs thereof.

19. A method of enhancing or inducing immunogenicity of a tumor cell in vivo, comprising:
obtaining a composition comprising a bispecific aptamer having a domain which specifically binds to a tumor antigen and a domain which specifically binds to an immune cell stimulatory molecule;
administering the aptamer composition in a therapeutically effective amount to the patient; and,
enhancing or inducing immunogenicity of a tumor cell.

20. The method of claim 19, wherein the aptamer is specific for a plurality of immune cell stimulatory molecules (multi-specificity).

21. The method of claim 19, wherein the aptamer is specific for a plurality of tumor antigens (multi-specificity).

22. The method of claim 19, wherein the aptamer is specific for a plurality of immune cell stimulatory molecules and at least one tumor antigen.

23. The method of claim 19, wherein the aptamer is specific for at least one immune stimulatory molecule and a plurality of tumor antigens.

24. The method of claim 19, wherein the composition comprises a plurality of aptamers with mono-specificity for an immune cell stimulatory and mono-specificity for a tumor antigen.

25. The method of claim 19, wherein the aptamer is specific for immune cell stimulatory molecules comprising at least one of: 4-1BB (CD 137), B7-1/2, 4-1BBL, OX40L, CD40, LIGHT, OX40, CD2, CD3, CD4, CD8a, CD11a, CD11b, CD11c, CD19, CD20, CD25 (IL-2Rα), CD26, CD27, CD28, CD40, CD44, CD54, CD56, CD62L (L-Selectin), CD69 (VEA), CD70, CD80 (B7.1), CD83, CD86 (B7.2), CD95 (Fas), CD134 (OX-40), CD137, CD137L, (Herpes Virus Entry Mediator (HVEM), TNFRSF14, ATAR, LIGHTR, TR2), CD150 (SLAM), CD152 (CTLA-4), CD154, (CD40L), CD178 (FasL), CD209 (DC-SIGN), CD 270, CD277, AITR, AITRL, B7-H3, B7-H4, BTLA, HLA-ABC, HLA-DR, ICOS, ICOSL (B7RP-1), NKG2D, PD-1 (CD279), PD-L1 (B7-H1), PD-L2 (B7-DC), TCR-α, TCR-β, TCR-γ, TCR-δ, ZAP-70, lymphotoxin receptor (LTβ), NK1.1, HLA-ABC, HLA-DR, T Cell receptor αβ (TCRαβ), T Cell receptor γδ (TCRγδ), T cell receptor ζ (TCRζ), TGFβRII, TNF receptor, Cd11c, CD1-339, B7, Foxp3, mannose receptor, or DEC205, variants, mutants, species variants, ligands, alleles and fragments thereof.

26. The method of claim 19, wherein the aptamer is specific for at least one tumor antigen comprising: PSMA; BRCA1, BRCA2, alpha-actinin-4; BCR-ABL fusion protein (b3a2); CASP-8; β-catenin; Cdc27; CDK4; dek-can fusion protein; Elongation factor 2; ETV6-AML1 fusion protein; LDLR-fucosyltransferase AS fusion protein; hsp70-2; KIAAO205; MART2; MUM-1f; MUM-2; MUM-3; neo-PAP; Myosin class I; OS-9g; pml-RAR alpha fusion protein; PTPRK; K-ras; N-ras; CEA; gp100/Pmel17; Kallikrein 4; mammaglobin-A; Melan-A/MART-1; PSA; TRP-1/gp75; TRP-2; tyrosinase; CPSF; EphA3; G250/MN/CAIX; HER-2/neu; Intestinal carboxyl esterase; alpha-fetoprotein; M-CSF; MUC1; p53; PRAME; RAGE-1; RU2AS; survivin; Telomerase; WT1; or CA125.

27. The method of claim 19, wherein a plurality of aptamer comprising compositions bind to tumor antigens and immune cell stimulatory molecules.

28. A method of specifically associating two or more cells in vitro or in vivo, comprising:
contacting at least one cell with a bi- and/or multi-specific aptamer molecule, wherein the aptamer specifically binds to a first target cell and a second target cell; and,
specifically associating two or more cells.

29. The method of claim 28, wherein the aptamer is specific for one or more molecules expressed by a first target cell.

30. The method of claim 28, wherein the aptamer is specific for one or more molecules expressed by a second target cell.

31. The method of claim 28, wherein the aptamer specifically binds to two or more target cells.

32. A method of modulating immune responses to vaccines comprises administration to a patient in vivo of an antigen specific vaccine and one or more bi-specific or multi-specific aptamers.

33. The method of claim 32, wherein the bi-specific or multi-specific aptamers activates a co-stimulatory receptor as compared to a normal control.

34. The method of claim 32, wherein the immune response against a specific antigen is increased as compared to a normal control.

35. A method of delivering a therapeutic molecule to a desired target cell in vivo comprising
contacting a cell with a composition comprising an aptamer and a therapeutic molecule wherein the aptamer specifically binds a target cell; and,
delivering a therapeutic molecule to a desired target cell.
